(19) United States
(12) Patent Application Publication
(10) Pub. No.: US 2008/0166432 A1
(43) Pub. Date: Jul. 10, 2008

(54) Biotherapeutics for Mitigation of Health Disorders from Terminalia Arjuna

(75) Inventors: Villoo Morawala Patell, Karnataka (IN); Dhruvdev Vyas, Karnataka (IN)

Correspondence Address: HAMRE, SCHUMANN, MUELLER & LARSON, P.C., P.O. BOX 2902, MINNEAPOLIS, MN, 55402-0902, US

(73) Assignee: Avestha Gengraine Technologies Pvt. Ltd., Bangalore (IN)

(21) Appl. No.: 10/568,521

(22) PCT Filed: Aug. 15, 2003
(86) PCT No.: PCT/IB03/03678

§ 371 Date: Feb. 13, 2007

(51) Int. Cl. A61K 36/00; A61P 3/10; A61P 9/00; A61P 35/00

(52) U.S. Cl. 424/725

(57) ABSTRACT

The invention relates to extracts from Terminalia plant species that are capable of being used in methods for managing diseases such as cardiovascular disease, diabetes, degenerative neurological diseases, cancer, age related diseases like amyloidosis, acute pancreatitis, arthritis, atherosclerosis, cancer, heart diseases, inflammatory bowel disease, myocardial infarction, senile dementia, retinal degeneration and senile cataract; owing to the extracts antioxidation potential.

The invention also relates to extracts from Terminalia plant species that are capable of being used in methods for managing various infectious diseases.

More particularly, the invention relates to certain extracts from Terminalia arjuna, their uses as antimicrobial and antioxidants agents for the treatment of certain diseases heart disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract in mammals, particularly humans, processes for obtaining them and delivery formats therefore

### FIELD OF INVENTION

The invention relates to extracts from Terminalia plant species that are capable of being used in methods for managing diseases such as cardiovascular disease, diabetes, degenerative neurological diseases, cancer, age related diseases like amyloidosis, acute pancreatitis, arthritis, atherosclerosis, cancer, heart diseases, inflammatory bowel disease, myocardial infarction, senile dementia, retinal degeneration and senile cataract; owing to the extracts antioxidation potential.

The invention also relates to extracts from Terminalia plant species that are capable of being used in methods for managing various infectious diseases.

More particularly, the invention relates to certain extracts from Terminalia arjuna, their uses as antimicrobial agents and antioxidants for the treatment of certain diseases like cardiovascular disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract in mammals, particularly humans, processes for obtaining them and delivery formats therefore.

### BACKGROUND

### Antioxidant Potential

Reactive oxygen species (ROS) are a family of molecules including molecular oxygen and its derivatives produced in all aerobic cells. Excessive production of ROS, outstripping endogenous antioxidant defense mechanisms, has been implicated in processes in which they oxidize biological macromolecules, such as DNA, protein, carbohydrates, and lipids. This condition has commonly been referred to as oxidant stress. An increasing body of evidence suggests that oxidant stress is involved in the pathogenesis of many cardiovascular diseases, including hypercholesterolemia, atherosclerosis, hypertension, diabetes, and heart failure.

Many ROS possess unpaired electrons and thus are free radicals. These include molecules such as superoxide anion (O₂⁻), hydroxyl racial (HO⁻), nitric oxide (NO⁻), and lipid radicals. Other reactive oxygen species, such as hydrogen peroxide (H₂O₂), peroxynitrite (ONOO⁻), and hypochlorous acid (HOCl), are not free radicals per se but have oxidizing effects that contribute to oxidant stress. The cellular production of one ROS may lead to the production of several others via radical chain reactions. For example, reactions between radicals and polyunsaturated fatty acids within cell membrane may result in a fatty acid peroxyl radical (R—COO⁻) that can attack adjacent fatty acid side chains and initiate production of other lipid radicals. Lipid radicals produced in this chain reaction accumulate in the cell membrane and may have a myriad of effects on cellular function, including leakage of the plasmolemma and dysfunction of membrane-bound receptors. Of note, end products of lipid peroxidation, including unsaturated aldehydes and other metabolites, have cytotoxic and mutagenic properties.

In mammalian cells, potential enzymatic sources of ROS include the mitochondrial respiration, arachidonic acid pathway enzymes lipoxygenase and cyclooxygenase, cytochrome p450s, xanthine oxidase, NADH/NADPH oxidases, NO synthase, peroxidases, and other hemoproteins. In addition to endogenous oxidative stress, exposure to free radicals and oxidants in the environment, such as ultraviolet sunlight, ozone, cigarette smoke, smog, and other pollutants, also contribute substantially to the rate of change in the body's oxidant: antioxidant balance. A shift in the oxidant: antioxidant balance due to increased production of free radicals may contribute to the decline of cardiovascular, neuronal, muscular, visual, and immune functions, over time. In addition, a high level of oxidative stress and free radicals has been implicated in an ever-widening array of age-related diseases like amyloidosis, acute pancreatitis, arthritis, atherosclerosis, cancer, cardiovascular disease, inflammatory bowel disease, myocardial infarction, senile dementia, retinal degeneration and senile cataract. (Am J Clin Nutr 2000; 71 (supll): 1665S-8S.)

Detoxification of ROS by antioxidants therefore affords protection against such diseases. There is a growing body of evidence suggesting that antioxidants contribute to cardioprotection.

Atherosclerosis, a chronic inflammatory disease of the arterial wall, is a major cause of morbidity and mortality from cardiovascular disease (CVD) in much of the world's population. Atherosclerosis is a complex process that leads to heart attack, stroke, and limb loss by the plugging of the arteries with atherosclerotic plaque. There have been several reports indicating oxidation of Low Density Lipoprotein (LDL) as one of the major mechanisms responsible for the pathogenesis of atherogenesis. The hypothesis that oxidative stress plays a role in atherosclerosis rests on the inference based on experimental work, on a large scale, carried out in animal models of heart disease and by extension, antioxidants by their ability to quench free radicals and reactive oxygen species, may play a beneficial role in modulating oxidative damage and thereby decreasing the risk of atherosclerotic lesion formation and progression. (J. Nutr. 131: 366S-368S, 2001.)

Nitric oxide (NO) is produced from L-arginine in the vascular endothelium by the endothelial iso-form of nitric-oxide synthase (NOS). Endothelial production of NO is crucial in the control of vascular tone, arterial pressure, smooth muscle cell proliferation and platelet adhesion to the endothelial surface. Impaired endothelium-derived NO bioactivity is a common feature of many vascular diseases that is thought to contribute to their clinical manifestations, as evidenced in a study conducted to investigate the effect of ascorbic acid on NO synthesis. The study also revealed that ascorbic acid was shown to enhance impaired endothelium-dependent vasodilatation in patients with atherosclerosis by a mechanism that is thought to involve protection of NO from inactivation by free oxygen radicals. Ascorbate pretreatment on endothelial cells led to a 3-fold increase of the cellular production of NO measured as the formation of its co-product citrulline and as the accumulation of its effector molecule cGMP. It was thus shown that intracellular ascorbic acid enhances NO synthesis in endothelial cells and that this may explain, in part, the beneficial vascular effects of ascorbic acid. (J. Biol. Chem. Vol. 274, No. 12, Issue of March 19, pp. 8254-8260, 1999, J. Biol. Chem. Vol. 275, No. 23, Issue of June 9, pp. 17399-17406, 2000.)

Degenerative neurological diseases affect millions of people around the world. A number of these diseases, including amyotrophic lateral sclerosis (ALS, or Lou Gehrig's disease), Parkinson's disease, and Alzheimer's disease, appear to have ROS toxicity as a central component of their underlying mechanism of nerve cell destruction. Unfortunately, there is little evidence that simply eating more dietary or even pharmocologic antioxidants will prevent or arrest the neural degeneration; not surprisingly the mechanism is too complex to lend itself to such a simplistic remedy. Nevertheless, improving our understanding of these complex injury mechanisms offers a real potential for improved clinical outcomes in the near future.

Ischemia/reperfusion injury is a particularly fascinating example of ROS-mediated disease. When an organ is deprived of its blood supply (ischemia) it is injured, not just by the temporary loss of oxygen, but also by the ROS that are generated by reaction with the oxygen that is reintroduced at reperfusion, when the blood supply is restored. In some clinical situations, we can prevent this injury by giving antioxidants, sometimes even after the period of ischemia, but just prior to reperfusion. For example, the preservation of kidneys, livers, and other organs in solutions that contain antioxidants, as well as other agents, is now routine prior to their transplantation. Another example is the use of drugs that block the function of free radical generating enzymes prior to stopping the heart for cardiac surgery. These drugs help prevent reperfusion injury when the heart is restarted and flow is restored. This reperfusion injury mechanism also has been found to play an important role in patients suffering from multiple organ failure after trauma, massive surgery, or shock. Multiple organ failure is now the leading cause of death in intensive care units, and extensive efforts are under way to understand better how ROS contribute to this syndrome.

Aging is a process per se, i.e., a series of controlled mechanisms, and not just the passive accumulation of wear and tear over the years. Put simply, our bodies age in the ways that are far more complex and more regulated than the processes by which our automobiles wear out. But if aging is a series of processes, it's logical to conclude that it is potentially controllable, or at least modifiable. One of the most important of these processes is comprised of an accumulation of the molecular injuries that are mediated by free radicals and other ROS. For example, since structural lipids are the primary component of our cell membranes, the integrity of which defines cell viability, aging is partially a matter of our going rancid as our lipids are progressively oxidized. While this is an oversimplification of this complex process, it reflects the optimism of some investigators of the aging process.

Recent studies indicate that the therapeutic manipulation of ROS metabolism can actually extend the total life span of mice to a significant degree. This was the first time that life span has been successfully altered experimentally by treatment. When one considers that the demographic, and consequent social, economic, and ecological impacts of even a 10 percent increase in human life span, a likely eventuality within the next decade or two, would far exceed that of a 100 percent cure for cancer (which is far less likely), the importance of this potential becomes evident.

As the understanding has evolved, it would provide unprecedented opportunities for improving the quality and even the length of human life.

### Antibacterial Potential

Resistance to existing drugs is developing at an alarming rate. Thus, a diverse arsenal of new antibacterial agents is urgently needed to combat the diminishing efficacy of existing antibiotics.

In India, herbal medicines have been the basis of treatment and cure for various diseases/physiological conditions in traditional methods. Although reports of antibacterial activity of indigenous plants have been published from many regions, they have not been systematically conducted, except in a few cases.

Terminalia arjuna Plant Extracts

Terminalia arjuna is a deciduous tree found throughout India growing to a height of around 60-90 feet. Terminalia arjuna belongs to the family Combretaceae and is called “Arjuna” in vernacular. Terminalia arjuna has been used for over 1500 years in India as a cardio tonic and has been indicated for derangement of all three humoursin, vata, pitta and kapha in Ayurveda. The bark of Terminalia arjuna has been widely used in Indian system of medicine for a variety of purposes.

Sharma V N et al. evaluated the antioxidant and hypocholesterolaemic effects of Terminalia arjuna tree bark (a popular cardiotonic substance in Indian pharmacopoeia) and compared it with a known antioxidant, vitamin E by a randomised controlled trial. It was concluded from this trial that, Terminalia arjuna tree bark powder has significant antioxidant action that is comparable to vitamin E. In addition, it also has a significant hypocholesterolaemic effect. (Antioxidant and hypocholesterolaemic effects of Terminalia arjuna tree-bark powder: a randomised placebo-controlled trial, J Assoc Physicians India 2001 February; 49:231-235)

The bark of Terminalia arjuna tree has a long history of use as a cardiac tonic as well, and has been indicated in the treatment of coronary artery disease, heart failure, hypercholesterolemia and for relief of anginal pain. (Miller, A. L. Botanical Influences on cardiovascular disease. Alternative Medicine Review. December 1998, vol 3. No. 6, pages 421-431.

Ethanolic extract of Terminalia arjuna tree bark in doses of 100 mg/kg and 500 mg/kg significantly reduced total and LDL cholesterol levels in hypercholesterolaemic rabbits. (Ram et al. Hypocholesterolaemic effects of Terminalia arjuna tree bark. Journal of Ethnopharmacology. Vol 55. No. 3, pages 165-169.)

It is reported that the bark of T. arjuna exhibited antibacterial activity only in dichloromethane, methanol, and aqueous extracts against E. coli, K. aerogenes, P. vulgaris, P. aerogenes at 1000-5000 ppm dosage. But there is no reference to the antibacterial activity of Ethyl acetate extract and other solvent extracts than mentioned above. Also there are no reports of the effect of Terminalia arjuna bark extracts on Gram positive bacteria. Additionally there are no reports of the effect of Terminalia arjuna fruit extracts on gram positive or gram negative bacteria (Samy et. al. Screening of 34 Indian medicinal plants for antibacterial properties. Journal of Ethanopharmacology 62 (1998) 173-182.).

It is reported that the bark of T. arjuna exhibited antioxidant activity only in direct aqueous extract as determined invitro by DPPH radical scavenging and deoxyribose damage protection assay and invivo by effect on lipid peroxidation. In the present invention direct and successive extracts except direct aqueous extract of T. arjuna bark and fruit have shown potent antioxidation activity (Munasinghe et. al., Antiradical and Antilipoperoxidative Effects of Some Plant Extracts used by Sri Lankan Traditional Medicinal Practitioners for Cardioprotection. Phytotherapy Research 15 (2001) 519-523).

There exists a need for the development of new medicines, which are effective in treating diseases like cardiovascular disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract.

### SUMMARY OF THE INVENTION

Objects of the invention will become apparent from the following description and examples.

The invention relates to extracts from Terminalia plant species that are capable of being used in methods for managing diseases such as heart disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract; owing to the extracts antioxidation potential.

The invention also relates to extracts from Terminalia plant species that are capable of being used in methods for managing various infectious diseases.

More particularly, the invention relates to certain extracts from Terminalia arjuna, their uses as antimicrobial agents and antioxidants for the treatment of certain diseases heart disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract in mammals, particularly humans, processes for obtaining them and delivery formats therefore.

### BRIEF DESCRIPTION OF THE EXTRACT NOMENCLATURE

### Nomenclature of Plant Extracts

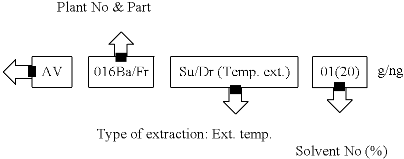 [0029] 1. AV—first two letters represents Avesthagen.
2. Plant Name: The Plants used and in use are assigned with unique 3-digit number, 016 represents Terminalia arjuna.
3. Part of the plant/Tissue: There is a two letter ID for each plant part used. For example Ba for Bark, Fr for whole Fruit.
4. Solvents: The solvents used for extraction are also assigned with two digit numbers 01 for Acetone, O₂ for Benzene, 03 for Chloroform, 04 for Ethanol, 05 for Hexane, 06 for Methanol, 07 for Petroleum ether, 08 for water, 09 for ethyl acetate. Percentage of solvents used for extraction is given within bracket (20) for 20% of that solvent. For example if 20% of Ethanol was used for extraction, 04(20).
5. Method of Extraction: Successive extraction is referred to as Su whereas direct extraction is referred to as Di, temperature for extraction is written in bracket. For example, Su(65) represents successive extraction at 65° C.
6. Type of extract, g: gluey and ng: non-gluey.

### BRIEF DESCRIPTION OF THE TABLES AND FIGURES

Table 1: HPLC fingerprint of the extract AV016BaDi(65)04(100).

Table 2: HPLC fingerprint of the extract AV016BaDi(28)04(20).

Table 3: HPLC fingerprint of the extract AV016BaSu(65)09(100).

Table 4: HPLC fingerprint of the extract AV016BaSu(65)01 (100).

Table 5: HPLC fingerprint of the extract AV016BaSu(65)01 (100)ng.

Table 6: HPLC fingerprint of the extract AV016BaSu(65)01 (100)g.

Table 7: HPLC fingerprint of the extract AV016BaSu(65)04(100).

Table 8: HPLC fingerprint of the extract AV016BaSu(65)06(100).

Table 9: HPLC fingerprint of the extract AV016BaSu(105)08(100).

Table 10: HPLC fingerprint of the extract AV016Fr(105)08(100).

Table 11: LC/MS Fingerprint of extract AV016BaDi(28)04(20) (TIC Spectrum (Q1 +ve mode)

Table 12: LC/MS Fingerprint of extract AV016BaDi(28)04(20) (TIC Spectrum (Q1 −ve Mode)

Table 13: LC/MS Fingerprint of extract AV016BaDi(65)04(100) (TIC Spectrum (Q1 +ve mode)

Table 14: LC/MS Fingerprint of extract AV016BaDi(65)04(100) (TIC Spectrum (Q1 −ve Mode)

Table 15: LC/MS Fingerprint of extract AV016BaSu(65)09(100) (TIC Spectrum (Q1 +ve mode)

Table 16: LC/MS Fingerprint of extract AV016BaSu(65)01 (100) (TIC Spectrum (Q1 +ve mode)

Table 17: LC/MS Fingerprint of extract AV016BaSu(65)01 (100) (TIC Spectrum (Q1 −ve Mode)

Table 18. LC/MS Fingerprint of extract AV016BaSu(65)01(100)ng (TIC Spectrum (Q1 +ve mode)

Table 19. LC/MS Fingerprint of extract AV016BaSu(65)01 (100)g (TIC Spectrum (Q1 +ve mode)

Table 20. LC/MS Fingerprint of extract AV016BaSu(65)04(100) (TIC Spectrum (Q1 +ve mode)

Table 21. LC/MS Fingerprint of extract AV016BaSu(65)06(100) (TIC Spectrum (Q1 +ve mode)

Table 22. LC/MS Fingerprint of extract AV016BaSu(105)08(100) (TIC Spectrum (Q1 +ve mode)

Table 23. LC/MS Fingerprint of extract AV016FrDi(65)04(100) (TIC Spectrum (Q1 +ve mode)

Table 24. LC/MS Fingerprint of extract AV016FrSu(105)08(100) (TIC Spectrum (Q1 +ve mode)

Table 25. IC₅₀ values of anti-oxidation activity of extracts from different T. arjuna plant parts

Table 26: Anti-bacterial activity of Terminalia arjuna bark successive extracts Table 27. Anti-bacterial activity of Terminalia arjuna fruit extracts:

FIG. 1: DPPH free radical scavenging potential of successive extracts of the bark of Terminalia arjuna

FIG. 2: DPPH free radical scavenging potential of successive extract of Terminalia arjuna bark with acetone solvent. [AV016BaSu(65)01 (100)g and AV016BaSu(65)01 (100)ng].

FIG. 3: DPPH free radical scavenging potential of fruit extracts of Terminalia arjuna with direct ethanol [AV016FrDi(65)04(100)] and successive water [AV016FrSu(105)08(100)] as solvents.

FIG. 4: DPPH free radical scavenging potential of direct extract of Terminalia arjuna bark with 100% ethanol solvent. [AV016BaDi(65)04(100)]

FIG. 5: DPPH free radical scavenging potential of direct extract of Terminalia arjuna bark direct with 20% ethanol solvent. [AV016BaDi(28)04(20)]

FIG. 6: Antibacterial activity of successive extract of Terminalia arjuna bark with ethyl acetate solvent. [AV016BaSu(65)09(100)].

FIG. 7. Antibacterial activity of successive extracts of Terminalia arjuna bark with acetone [AV016BaSu(65)01(100)], Ethanol [AV016BaSu(65)04(100)], Methanol [AV016BaSu(65)06(100)], Ethyl acetate [AV016BaSu(65)09(100)], and Water [AV016BaSu(105)08(100)] as solvents.

FIG. 8: Growth of the bacterial strains on the LB, LB with DMSO and LB with ciprofloxacin.

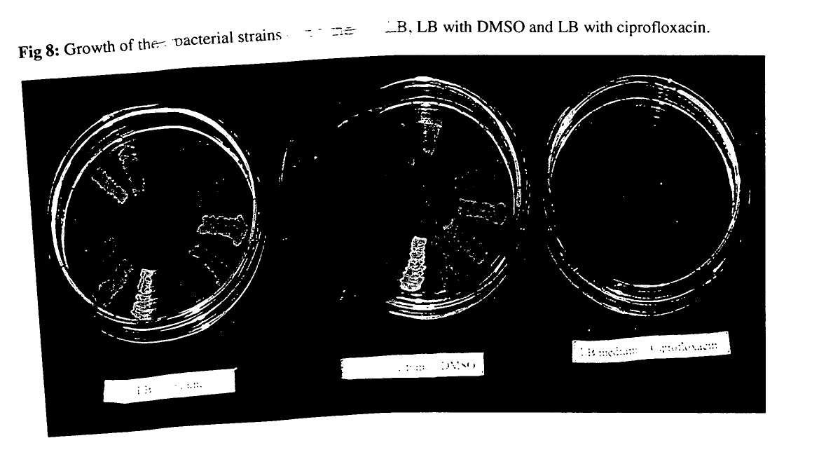

### DETAILED DESCRIPTION OF THE INVENTION

In a first aspect of the invention there is provided a method for treating a disease in a mammal, which comprises administering to the said mammal an effective non-toxic amount of at least an extract from Terminalia arjuna as defined herein. Preferably the mammal is a human being. The skilled addressee will appreciate that “treating a disease” in a mammal means treating, that is to say, alleviating symptoms of the disease and may also mean managing a disease in the sense of preventing such a disease state either advancing ie getting worse or becoming more invasive, or slowing down the rate of advance of a disease.

In a second aspect of the invention, there is a provided a prophylactic method for preventing the occurrence of a disease state in a mammal which comprises administering to the said mammal an effective non-toxic amount of an extract from Terminalia arjuna as defined herein in the preparation of a comestible (=foodstuff) for prophylaxis against the occurrence of a disease diseases like cardiovascular disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract. Preferably the mammal is human and the said extract comprises a single extract from a plant part of Terminalia arjuna or a combination of extracts therefrom as detailed herein. Thus the present invention further relates to extracts which may be isolated from different parts of the Terminalia arjuna plant such as the bark and fruit thereof, the preparation of such extracts, medicaments comprising such extracts, and the use of these extracts and constituents for the preparation of a medicament.

Extracts of the present invention can be isolated from Terminalia tree species, such as Terminalia arjuna, using conventional organic solvent extraction and supercritical fluid extraction technology. Generally, extracts of the invention capable of functioning in a prophylactic or therapeutic manner as outlined herein can be extracted from any Terminalia arjuna plant tissue, such as bark or fruit, depending on the end purpose that is required of the extract.

In a third aspect of the present invention there is provided a process for preparing extracts of the invention from plant parts of Terminalia arjuna that comprises:
Pulverizing selected plant material to a powder;
Subjecting the powdered plant material to solvent extraction;
Lyophilizing the obtained extracts.

The choice of selected plant material may be of any type but is preferably selected from the bark or the fruit of the Terminalia arjuna plant.

The solvent extraction process may be selected from direct or successive extraction types such as extraction from plant parts in soxhlet apparatus or in flasks at room temperature or at higher temperature with polar and/or non-polar solvent(s). Typically, the extraction process is as outlined herein.

It will be apparent to the skilled addressee that the selection of solvent, or mixtures of solvents for each step in the isolation of extracts of the invention showing activity can be guided by results of bioassay analysis of separate fractions, for example as indicated herein and/or as shown in the examples.

Also encompassed within the ambit of the invention is a pharmaceutical formulation suitable for use in the treatment of a disease selected from the group heart disease, diabetes, degenerative neurological diseases, cancer, age related diseases like amyloidosis, acute pancreatitis, arthritis, atherosclerosis, cancer, cardiovascular disease, inflammatory bowel disease, myocardial infarction, senile dementia, retinal degeneration and senile cataract; comprising at least one extract as isolated from a Terminalia species, such as Terminalia arjuna, in admixture with a pharmaceutically acceptable carrier. Preferably, the at least one extract is selected from those listed in Tables 1-24 inclusive, depending on design and disease of interest. Preferably the at least one extract is selected from the group of extracts as defined in Tables 25-27 inclusive, again depending on end purpose. Naturally, the skilled addressee will appreciate that such compositions may comprise of two or more plant extracts of the invention in any concentration, which is capable of giving rise to a therapeutic effect. Thus, therapeutic compositions can comprise plant extracts of Terminalia substantially devoid of undesirable contaminating compounds. The plant extracts may have, for example, undergone a number of solvent extraction steps substantially to separate out undesirable components from desirable components such as those alluded to in the examples and aforementioned tables.

The invention thus further provides a method for the treatment of a disease selected from the group heart disease, diabetes, degenerative neurological diseases, cancer, age related diseases like amyloidosis, acute pancreatitis, arthritis, atherosclerosis, cancer, cardiovascular disease, inflammatory bowel disease, myocardial infarction, senile dementia, retinal degeneration and senile cataract; in mammals, including humans, which comprises the use of a clinically useful amount of an extract selected from those listed in Tables 1-24 inclusive, preferably those listed in Tables 25-27 inclusive, in a pharmaceutically useful form, once or several times a day or in any other appropriate schedule for example, orally, or intravenously or by delivery to the lungs in a dry or “wet” spray.

The amount of compound of extract required to be effective in the treatment of the aforementioned diseases will, of course, vary with the disease being treated and is ultimately at the discretion of the medical or veterinary practitioner. The factors to be considered include the condition being treated, the route of administration, and nature of the formulation, the mammal's body weight, surface area, age and general condition and the particular compound to be administered. A suitable effective dose of an extract of the invention generally lies in the range of about 0.01 to about 120 mg/kg bodyweight, e.g. 0.1 to about 120 mg/kg body weight, preferably in the range of about 0.1 to 50 mg/kg, for example 0.5 to 50 mg/kg. The total daily dose may be given as a single dose, multiple doses, e.g. two to six times applications per day. For example, for a 75 kg mammal (e.g. a human) the dose range would be about 8 to 9000 mg per day, and a typical dose could be about 50 mg per day. If discrete multiple doses are indicated treatment might typically be 15 mg of a compound of Formula (I) given up to 4 times per day.

Whilst it is possible for the active extract to be administered alone, it is preferred to present the active extract in a pharmaceutical formulation. Formulations of the present invention, for medical use, comprise an extract of the invention together with one or more pharmaceutically acceptable carriers and optionally other therapeutic ingredients. The carrier(s) should be pharmaceutically acceptable in the sense of being compatible with the other ingredients of the formulation and substantially non-deleterious to the recipient thereof.

The present invention, therefore, further provides a pharmaceutical formulation comprising at least one extract selected from those listed in tables 1-24 inclusive, preferably from those mentioned in tables 25-27 inclusive together with a pharmaceutically acceptable carrier therefore. In a preferment the pharmaceutical formulation comprises at least an extract selected from those listed in tables 25-27, depending on the disease type being treated. Naturally, the skilled addressee will appreciate that when selecting more than one extract from those given in the aforementioned tables for the treatment of any single disease type, that an appropriate selection of extracts from the disease type will be made. Thus, for example, for the treatment of diabetes, extracts appropriate for doing so will be selected from the said tables.

Naturally, the skilled addressee will appreciate that any pharmaceutical formulation comprising an active extract of the invention can include at least one active extract purified from an extract derived from a Terminalia species. Thus a pharmaceutical formulation may contain more than one active extract derived from two or more Terminalia species.

There is also provided a method for the preparation of a pharmaceutical formulation comprising bringing into association an extract of the invention, and a pharmaceutically acceptable carrier therefore.

Formulations according to the present invention include those suitable for oral or intravenous administration.

Intravenous formulations including at least one extract of the invention and may also be administered in the form of suitable liposomal or niosomal preparations or other suitable delivery vehicle.

Emulgents and emulsion stabilizers suitable for use in the formulation of the present invention include Tween 60, Span 80, cetostearyl alcohol, myristyl alcohol, glycerol mono-stearate and sodium laury sulphate.

The formulations may conveniently be presented in unit dosage form and may be prepared by any of the methods well known in the art of pharmacy. All methods include the step of bringing the active extracts(s) into association with a carrier which constitutes one or more accessory ingredients. In general, the formulations are prepared by uniformly and intimately bringing the active extract(s) into association with a liquid carrier or a finely divided solid carrier or both and then, if necessary, shaping the product into desired formulations.

Formulations of the present invention suitable for oral administration may be presented as discrete units such as capsules, sachets, tablets, lozenges, comprising the active ingredient in a flavoured based, usually sucrose and acacia or tragacanth; pastilles comprising the active ingredient in an inert base such as gelatin and glycerin, or sucrose and acacia; and mouth-washes comprising the active ingredient in a suitable liquid carrier. Each formulation generally contains a predetermined amount of the active extract; as a powder or granules; or a solution or suspension in an aqueous or non-aqueous liquid such as a syrup, an elixir, an emulsion or draught and the like.

A tablet may be made by compression or moulding optionally with one or more accessory ingredients. Compressed tablets may be prepared by compressing an a suitable machine the active extract in a free-flowing form such as a powder or granules, optionally mixed with a binder, (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface active or dispersing agent. Moulded tablets may be made by moulding in a suitable machine a mixture of the powdered extract moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

A syrup may be made by adding the active extract to a concentrated, aqueous solution of a sugar, for example sucrose, to which may also be added any necessary ingredients. Such accessory ingredient(s) may include flavourings, an agent to retard crystallisation of the sugar or an agent to increase the solubility of any other ingredients, such as a polyhydric alcohol for example glycerol or sorbitol.

In addition to the aforementioned ingredients, the formulations of this invention may further include on or more accessory ingredients) selected from diluents, buffers, flavouring agents, binders, surface active agents, thickeners, lubricants, preservatives (including antioxidants) and the like.

Alternatively, the compositions are dietary supplements, food compositions or beverage compositions suitable for human or animal consumption.

The invention describes the HPLC profiles and Mass spectrums of direct and successive solvent extracts of Terminalia arjuna plant parts thereby giving each extract an identity of itself. The various solvents used for successive extraction are in order from non-polar to polar side i.e hexane, petroleum ether, ethyl acetate, acetone, ethanol, methanol and water. In case of direct extraction alcoholic solvent alone and in combination with water was used as solvent for extraction.

The invention further encompasses novel extracts defined by reference to their HPLC and MS fingerprints as defined in Tables 1-24 inclusive, which are isolated from different parts of Terminalia arjuna plant, the preparation of such extracts, the medicaments containing said extracts, and the use of these extracts and constituents for the preparation of a medicament.

In one embodiment of the invention, the compositions for preventing, treating, or managing diseases such as heart disease, diabetes, degenerative neurological diseases, cancer, age related disease like amyloidosis, acute pancreatitis, arthritis, atherosclerosis, cancer, cardiovascular disease, inflammatory bowel disease, myocardial infarction, senile dementia, retinal degeneration and senile cataract comprises of direct extracts of T. arjuna bark with 100% ethanol solvent [AV016BaDi(65)04(100)] and 20% ethanol solvent [AV016BaDi(28)04(20)], successive extract of T. arjuna bark with ethyl acetate solvent [AV016BaSu(65)09(100)], successive extract of T. arjuna bark with acetone solvent [AV016BaSu(65)01 (100)], [AV016BaSu(65)01 (100)g] and [AV016BaSu(65)01(100)ng], successive extract of T. arjuna bark with ethanol solvent [AV016BaSu(65)04(100)], successive extract of T. arjuna bark with methanol solvent [AV016BaSu(65)06(100)] and successive extract of T. arjuna bark with water solvent [AV016BaSu(105)08(100)], direct extract of T. arjuna fruit with ethanol solvent [AV016FrDi(65)04(100)] and successive extract of T. arjuna fruit with water solvent [AV016FrSu(105)08(100)], alone or in combination thereof. The compositions/medicaments may contain a pharmaceutically acceptable carrier, excipient, or diluent.

In another embodiment of the invention, the compositions for preventing, treating, or managing microbial infections comprises of successive extract of T. arjuna bark with ethyl acetate solvent [AV016BaSu(65)09(100)], successive extract of T. arjuna bark with acetone solvent [AV016BaSu(65)01(100)], successive extract of T. arjuna bark with ethanol solvent [AV016BaSu(65)04(100)], successive extract of T. arjuna bark with methanol solvent [AV016BaSu(65)06(100)] and successive extract of T. arjuna bark with water solvent [AV016BaSu(105)08(100)], direct extract of T. arjuna fruit with ethanol solvent [AV016FrDi(65)04(100)] and successive extract of T. arjuna fruit with water solvent [AV016FrSu(105)08(100)], alone or in combination thereof. The compositions/medicaments may contain a pharmaceutically acceptable carrier, excipient, or diluent.

In a further aspect of the invention there is provided a comestible, that is to say, a foodstuff comprising at least an extract of the invention, typically in dried form, such as in a lyophilized form selected from those listed in Tables 1-24 herein, and in particular, from those extracts selected from those mentioned in Tables 25-27. The skilled addressee will appreciate that such comestibles may contain more than one extract of the invention and may be used. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, for example that of having a prophylactic effect against the occurrence of diabetes, or a comestible may have a multi-functional prophylactic effect against two or more disease types. It is thought that a similar multi-functional role could also be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectioneries, baked products including breads such as loafs, and flat breads such as pitta bread, naan bread and the like, cakes, snack foods such as muesli bars, compressed dried fruit bars, biscuits, dairy products such as yoghurts, milk and milk-based products such as custards, cream, cheese, butter and crème fraiche, simulated dairy food products such as margarine, olive oil-based spreads, and low fat cream substitutes such as Elmlea products, fruit and vegetable juices, aerated drinks, such as carbonated soft drinks and non-aerated drinks such as squashes, soya milk, rice milk and coconut milk and the like, pastas, noodles, vegetable, seed and nut oils, fruited oils such as sunflower oil, rapeseed oil, olive oil, walnut, hazelnut, and sesame seed oil and the like, and frozen confections such as ice creams, iced yoghurts and the like.

A suitable effective dose of an extract of the invention to be included in a comestible generally lies in the range of about 0.01 to about 120 mg/kg bodyweight, e.g. 0.1 to about 120 mg/kg body weight, preferably in the range of about 0.1 to 50 mg/kg, for example 0.5 to 50 mg/kg. The total daily dose may be given as a single dose, multiple doses, e.g. two to six times applications per day.

In a further aspect of the invention there is provided use of an extract selected from those of Tables 1-24, and in particular those of Tables 25-30 for the preparation of a medicament for the treatment of a disease selected from the group consisting of heart disease, diabetes, degenerative neurological diseases, cancer, age related diseases like amyloidosis, acute pancreatitis, arthritis, atherosclerosis, cancer, cardiovascular disease, inflammatory bowel disease, myocardial infarction, senile dementia, retinal degeneration and senile cataract.

Thus, there is provided use of an extract selected from the group consisting of AV016BaDi(65)04(100), AV016BaDi(28)04(20), AV016BaSu(65)09(100), AV016BaSu(65)01 (100), AV016BaSu(65)01 (100)g, AV016BaSu(65)01(100)ng, AV016BaSu(65)04(1100), AV016BaSu(65)06(100), AV016BaSu(105)08(100), AV016FrDi(65)04(100) and AV016FrSu(105)08(100), alone or in combination thereof for the preparation of a medicament for the treatment or prophylaxis of such as cardiovascular disease, diabetes, degenerative neurological diseases, cancer, age related diseases like amyloidosis, acute pancreatitis, arthritis, atherosclerosis, cancer, heart diseases, inflammatory bowel disease, myocardial infarction, senile dementia, retinal degeneration and senile cataract; owing to the extracts antioxidation potential.

Thus, there is provided use of an extract selected from the group consisting of AV016BaSu(65)09(100), AV016BaSu(65)01(100), AV016BaSu(65)04(100), AV016BaSu(65)06(100), AV016BaSu(105)08(100), AV016FrDi(65)04(100)] and AV016FrSu(105)08(100)], alone or in combination thereof for the preparation of a medicament for the treatment or prophylaxis of infectious diseases, owing to the extracts antimicrobial potential.

The invention will now be exemplified with reference to the following Examples section and accompanying tables and Figures. It is to be understood that the examples are not to be construed as limiting the scope of the invention in any way.

The invention will now be exemplified with reference to the following Examples section and accompanying tables and Figures. It is to be understood that the examples are not to be construed as limiting the scope of the invention in any way.

### EXAMPLES SECTION

### Example 1

### Extraction of Terminalia arjuna

Extraction of Terminalia arjuna plant parts was carried out by both direct extraction as well as successive extraction method, at room temperature as well as in soxhlet apparatus and related liquid-liquid techniques followed by lyophilization.

### I. Successive Extraction.

Successive extraction from bark of Terminalia arjuna was carried out using soxhlet extractor. The solvents used, were based on their sequential polarity starting from non-polar to polar, viz; Hexane, chloroform, ethyl acetate, acetone, ethanol, methanol and water at 65° C./above boiling point of the solvent.

The detailed Process is given below:
1. Weigh 50 grams of powdered plant material into the extractor (Soxhlet extractor body) with the cotton on the bottom of the soxhlet apparatus. Cover with cotton on the top. Add 250 ml of solvent (start with Hexane) in to the round-bottomed flask and place it on the mantle and add few ceramic chips in to it. Add 50 ml of solvent over the material just wetting it.
2. Place the extractor on the flask, which is in turn connected with the condenser.
3. Let the cold water circulate continuously in the condenser from the tap.
The set up fits tightly as it is fabricated as one set.
4. Switch on the mantle and set it at 65° C. The vapors of the solvent from the flask would pass through the inlet of the extractor and will get condensed. The condensed (distilled) Solvent will get collected in the Extractor (body) thus extracting the compounds from it.
5. When the plant material is completely filled with solvents, it will get drained in the flask. This process is continuous as long as there is stable heat and water circulation.
6. Continue the extraction for 8 hours, 4-5 cycles per hour.
7. The extract collected in the flask is concentrated by vacuum lyophilization.
8. Follow the same procedure as above successively for the following solvents in the same order. Hexane, chloroform, Ethyl acetate, Acetone, Methanol and Water.

### II. Direct Extraction

a. Soxhlet Based Extraction Procedure with 100% Ethanol Solvent
1. Weigh 100 grams of powdered plant material in the cloth bag and transfer it into the extractor (Soxhlet extractor body). Cover with cotton on the top. (Make sure the level of material is below one inch of the vapour inlet tube.)
2. Add 1 liter of solvent (start with Pet. ether) in to the round-bottomed flask and place it on the mantle and add few ceramic chips in to it. Add 100 ml of solvent over the material just wetting it.
3. Place the extractor on the flask, which is in turn connected with the condenser.
4. Let the cold water circulate continuously in the condenser from the tap. The set up fits tightly as it is fabricated as one set.
5. Switch on the mantle and set it at 65° C. The vapours of the solvent from the flask would pass through the inlet of the extractor and will get condensed. The condensed (distilled) Solvent will get collected in the Extractor (body) thus extracting the compounds from it.
6. When the plant material is completely filled with solvents, it will get drained in the flask. This process is continuous as long as there is stable heat and water circulation.
7. Continue the extraction for 8 hours, 4-5 cycles per hour.
8. The extract collected in the flask is concentrated by vacuum lyophilization.
   b. Extraction of T. arjuna Bark with 20% Ethanol Solvent at Room Temperature:
1. Weigh known quantity (100 grams) of powdered plant material into the conical flask and cover the mouth with aluminum foil to avoid solvent evaporation.
2. Add known volume (500 ml) of 20% ethanol (100 ml ethanol+400 ml water) solvent in to the flask and place it on to the orbital shaker and set the speed at 210 rpm and room 28° C. temperature for the extraction.
3. Extract the plant material for 4 hr and drain the solvent through
4. Centrifuge the filtrate at 1000 rpm for 10 mins. Collect the supernatant and subject it to lyophilization.
5. Re-extract with 250 ml of solvent for (2×2 hrs).
6. Centrifuge the filtrate at 1000 rpm for 10 mins.
7. Concentrate extract using lyophilizer under vacuum.

### Example 2

### Metabolic Fingerprinting of the Terminalia arjuna Extracts

Metabolic fingerprinting of all the direct and successive extracts from Terminalia arjuna plant parts is done by HPLC and LC-MS technique.

### I. HPLC Fingerprinting:

The plant extracts obtained by direct/successive extraction are subjected to HPLC analysis. High Performance Liquid Chromatography (HPLC) is a technique wherein small quantity of the sample is injected into a C-18 column under high pressure and the constituents are allowed to separate based on their interaction with the column and their retention time within the column. The main purpose of HPLC analysis is to find out the total number of constituents in the plant extracts.

The samples are prepared for HPLC analysis by dissolving the appropriate weight of the extract in methanol. These samples are filtered and collected in. the total recovery HPLC vials. These samples are subjected to separation by Waters 2695 HPLC instrument and then analyzed at 250 nm.

### Run Conditions:

1. The software used for HPLC analysis is Waters Millennium³²
2. The HPLC column used for separation is Waters μBondpack C-18, 5μ, 4.6×150 mm.
3. Column temperature is maintained at 25° C.
4. Solvent flow rate is set at 1.0 ml per min. HPLC conditions included Gradient chromatography—solvents used are acetonitrile (solvent A), methanol (solvent B) and water (Solvent C and D).
   Terminalia arjuna Extracts and HPLC Run Conditions:
   1. Terminalia arjuna Extracts:

1. AV016BaDi(65)04(100)

2. AV016BaDi(28)04(20)

3. AV016BaSu(65)04(100)

4. AV016FrDi(65)04(100)

5. AV016BaSu(65)06(100)

### 1. Method Set: Ethanol_{—}11

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 1.00 | 10.0 | 0.0 | 0.0 | 90.0 | 6 |
| 2 | 1.00 | 1.00 | 10.0 | 0.0 | 0.0 | 90.0 | 6 |
| 3 | 15.00 | 1.00 | 30.0 | 0.0 | 0.0 | 70.0 | 6 |
| 4 | 30.00 | 1.00 | 40.0 | 0.0 | 0.0 | 60.0 | 6 |

A: Acetonitrile, B: Methanol, C: Water

2. Terminalia arjuna Extracts

1. AV016BaSu(65)01(100)

2. AV016BaSu(65)01(100)g

3. AV016BaSu(65)01(100)g

II. Method Set: Ethyl Acetate_{—}10a

Pressure Limits:
High Limits 4000 psi Low limits 0 psi

Programmed Flow:

Pump Mode Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 5.0 | 2.5 | 92.5 | 0.0 | 6 |
| 2 | 1.00 | 0.75 | 5.0 | 2.5 | 92.5 | 0.0 | 6 |
| 2 | 25.00 | 0.75 | 25.0 | 2.5 | 72.5 | 0.0 | 6 |
| 3 | 30.00 | 0.75 | 5.0 | 2.5 | 92.5 | 0.0 | 1 |

A: Acetonitrile, B: Methanol, C: Water

3. Terminalia arjuna extract:

1. AV016BaSu(65)09(100)

III. Method Set: Ethyl Acetate_{—}4a

Pressure Limits:

High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 0.0 | 5.0 | 95.0 | 0.0 | 6 |
| 2 | 1.00 | 0.75 | 0.0 | 5.0 | 95.0 | 0.0 | 6 |
| 3 | 15.00 | 0.75 | 0.0 | 20.0 | 80.0 | 0.0 | 6 |
| 4 | 25.00 | 0.75 | 0.0 | 50.0 | 50.0 | 0.0 | 6 |
| 5 | 30.00 | 0.75 | 0.0 | 5.0 | 95.0 | 0.0 | 1 |

### A: Acetonitrile, B: Methanol, C: Water

4. Terminalia arjuna Extract:

1. AV016BaSu(105)08(100)

2. AV016FrSu(105)08(100)

IV. Method Set: Gy_Water_{—}12

Pressure Limits:
High Limits 4000 psi Low limits 0 psi

Programmed Flow:
Pump Mode: Gradient
Accelerated to 10 ml/min in: 2.0 min (5 ml/min/min)

| | Time | Flow | % A | % B | % C | % D | Curve |
|---|---|---|---|---|---|---|---|
| 1 | 0.01 | 0.75 | 5.0 | 5.0 | 90.0 | 0.0 | 6 |
| 2 | 1.00 | 0.75 | 5.0 | 5.0 | 90.0 | 0.0 | 6 |
| 3 | 20.00 | 0.75 | 25.0 | 15.0 | 60.0 | 0.0 | 4 |
| 4 | 26.00 | 0.75 | 70.0 | 5.0 | 25.0 | 0.0 | 4 |
| 5 | 30.00 | 0.75 | 5.0 | 5.0 | 90.0 | 0.0 | 1 |

### A: Acetonitrile, B: Methanol, C: Water

### II. LC/MS Fingerprinting:

### II. Liquid Chromatography Mass Spectrometry (LC/MS) Fingerprinting:

Mass spectroscopy, is an instrumental approach that allows for the mass measurement of molecules. The five basic components of mass spectrometer are a vacuum system, a sample introduction device, an ionization source, a mass analyzer and an ion detector. Combining these parts a mass spectrometer determines the molecular weight of chemical compounds by ionizing, separating and measuring molecular ions according to their mass-to-charge ratio (m/z).

Run conditions used for LC/MS fingerprinting of Terminalia arjuna is shown down.
1. Q-Trap LC/MS instrument from Applied Biosystems was used. The software used for LC/MS analysis is Analyst
2. The HPLC column used for separation is COSMOSIL® 5C₁₈-MS-II Packed Column C-18, 5 μm, 4.6 mml.D.x 150 mm.
3. Column temperature is maintained at 25° C.
4. Solvent flow rate is set at 11.0 ml per min. HPLC conditions included Gradient chromatography—solvents used are acetonitrile (solvent C), methanol (solvent B) and water (Solvent D).
   1. Terminalia arjuna Extracts:

AV016BaSu(65)09(1100), AV016BaSu(65)01 (100), AV016BaSu(65)01(100)g, AV016BaSu(65)01(100)g, AV016BaSu(65)04(1100), AV016BaSu(65)06(1100), AV016BaDi(65)04(1100), and AV016FrDi(65)04(1100)

a. LC/MS Sample Run Conditions for all the above-mentioned Terminalia arjuna Samples:

| Mass Spectrometer QTrap | 0 | MASS SPEC |
|---|---|---|
| Config Table Ver | 01 | |
| Firmware Ver | M401400 B4T0301 M3L | |
| | 1400 B3T0300 | |
| Component Name | Linear Ion Trap | |
| | Quadrupole LC/MS/MS | |
| | Mass Spectrometer | |
| Component ID | QTrap | |
| Manufacturer | AB Sciex Instruments | |
| Model | 027170-C | |
| S/N | M1100301 | |
| Time from start = 0.0500 min | | |
| Mass Spectrometer QTrap | 0 | MASS SPEC |
| Start of Run - Detailed Status | | |
| Vacuum Status | At Pressure | |
| Vacuum Gauge (10e−5 Torr) | 0.7 | |
| Backing Pump | Ok | |
| Dual Turbo Pump | Normal | |
| Sample Introduction Status | Ready | |
| Source/Ion Path Electronics | On | |
| Source Type | Turbo Spray | |
| Source Temperature (at setpoint) | 400.0 C. | |
| Source Exhaust Pump | Ok | |
| Interface Heater | Ready | |
| Mass Spectrometer QTrap | 0 | MASS SPEC |
| End of Run - Detailed Status | | |
| Vacuum Status | At Pressure | |
| Vacuum Gauge (10e−5 Torr) | 0.7 | |
| Backing Pump | Ok | |
| Dual Turbo Pump | Normal | |
| Sample Introduction Status | Ready | |
| Source/Ion Path Electronics | On | |
| Source Type | Turbo Spray | |
| Source Temperature (at setpoint) | 400.0 C. | |
| Source Exhaust Pump | Ok | |
| Interface Heater | Ready | |
| Time from start = 61.4833 min | | |

### PE LC-200 Pump Method Properties

PE LC-200 Quaternary Pump

| | |
|---|---|
| Minimum Pressure (psi): | 0.0 |
| Maximum Pressure (psi): | 6100.0 |
| Shutdown Time (min): | 999.9 |

Step Table:

| | Total Time | Flow Rate | | | | | | | |
|---|---|---|---|---|---|---|---|---|---|
| Step | (min) | (μl/min) | GradientProfile | A (%) | B (%) | C (%) | D (%) | TE#1 | TE#2 |
| 0 | 0.5 | 1000.00 | 1.0 | 0.0 | 0.0 | 10.0 | 90.0 | open | open |
| 1 | 1.0 | 1000.00 | 1.0 | 0.0 | 0.0 | 10.0 | 90.0 | open | open |
| 2 | 15.0 | 1000.00 | 1.0 | 0.0 | 0.0 | 15.0 | 85.0 | open | open |
| 3 | 40.0 | 1000.00 | 1.0 | 0.0 | 0.0 | 25.0 | 75.0 | open | open |
| 4 | 50.0 | 1000.00 | 1.0 | 0.0 | 0.0 | 35.0 | 65.0 | open | open |
| 5 | 60.0 | 1000.00 | 1.0 | 0.0 | 0.0 | 50.0 | 50.0 | open | open |

### Quantitation Information:

| | |
|---|---|
| Sample Type: | Unknown |
| Dilution Factor: | 1.000000 |
| Custom Data: | |
| Quantitation Table: | |

### Period 1:

| | |
|---|---|
| Scans in Period: | 2243 |
| Relative Start Time: | 0.00 msec |
| Experiments in Period: | 1 |

### Period 1 Experiment 1:

| | |
|---|---|
| Scan Type: | Q1 MS (Q1) |
| Polarity: | Positive |
| Scan Mode: | Profile |
| Resolution Q1: | UNIT |
| Intensity Thres.: | 0.00 cps |
| Settling Time: | 0.0000 ms |
| MR Pause: | 5.0070 ms |
| MCA: | No |
| Center/Width: | No |
| Step Size: | 0.10 amu |

| Start (amu) | Stop (amu) | Time (sec) | Param | Start | Stop |
|---|---|---|---|---|---|
| 50.00 | 1700.00 | 1.60 | CEP | 6.47 | 66.65 |

### Parameter Table (Period 1 Experiment 1)

| | |
|---|---|
| CUR: | 20.00 |
| TEM: | 400.00 |
| GS1: | 20.00 |
| GS2: | 50.00 |
| ihe: | ON |
| IS: | 4500.00 |
| DP | 90.00 |
| EP | 10.00 |

2. Terminalia arjuna Extracts:

AV016BaSu(105)08(100), AV016FrSu(105)08(100) and AV016BaDi(28)04(20).

a. LCIMS Sample Run Conditions for all the above-mentioned Terminalia arjuna samples:

| Mass Spectrometer QTrap | 0 | MASS SPEC |
|---|---|---|
| Config Table Ver | 01 | |
| Firmware Ver | M401400 B4T0301 | |
| | M3L1400 B3T0300 | |
| Component Name | Linear Ion Trap | |
| | Quadrupole LC/MS/MS | |
| | Mass Spectrometer | |
| Component ID | QTrap | |
| Manufacturer | AB Sciex Instruments | |
| Model | 027170-C | |
| S/N | M1100301 | |
| Time from start = 2.1000 min | | |
| Mass Spectrometer QTrap | 0 | MASS SPEC |
| Start of Run - Detailed Status | | |
| Vacuum Status | At Pressure | |
| Vacuum Gauge (10e−5 Torr) | 0.7 | |
| Backing Pump | Ok | |
| Dual Turbo Pump | Normal | |
| Sample Introduction Status | Ready | |
| Source/Ion Path Electronics | On | |
| Source Type | Turbo Spray | |
| Source Temperature (at setpoint) | 400.0 C. | |
| Source Exhaust Pump | Ok | |
| Interface Heater | Ready | |
| Time from start = 2.1167 min | | |
| Mass Spectrometer QTrap | 0 | MASS SPEC |
| End of Run - Detailed Status | | |
| Vacuum Status | At Pressure | |
| Vacuum Gauge (10e−5 Torr) | 0.7 | |
| Backing Pump | Ok | |
| Dual Turbo Pump | Normal | |
| Sample Introduction Status | Ready | |
| Source/Ion Path Electronics | On | |
| Source Type | Turbo Spray | |
| Source Temperature (at setpoint) | 400.0 C. | |
| Source Exhaust Pump | Ok | |
| Interface Heater | Ready | |
| Time from start = 42.9333 min | | |

### PE LC-200 Pump Method Properties

PE LC-200 Quaternary Pump

| | |
|---|---|
| Minimum Pressure (psi): | 0.0 |
| Maximum Pressure (psi): | 6100.0 |
| Shutdown Time (min): | 999.9 |

Step Table:

| | Total Time | Flow Rate | | | | | | | |
|---|---|---|---|---|---|---|---|---|---|
| Step | (min) | (μl/min) | GradientProfile | A (%) | B (%) | C (%) | D (%) | TE#1 | TE#2 |
| 0 | 0.5 | 750.00 | 1.0 | 0.0 | 5.0 | 5.0 | 90.0 | open | open |
| 1 | 1.0 | 750.00 | 1.0 | 0.0 | 5.0 | 5.0 | 90.0 | open | open |
| 2 | 20.0 | 750.00 | 1.0 | 0.0 | 15.0 | 25.0 | 60.0 | open | open |
| 3 | 26.0 | 750.00 | 1.0 | 0.0 | 5.0 | 70.0 | 25.0 | open | open |
| 4 | 30.0 | 750.00 | 1.0 | 0.0 | 5.0 | 5.0 | 90.0 | open | open |
| 5 | 40.0 | 750.00 | 1.0 | 0.0 | 5.0 | 5.0 | 90.0 | open | open |

### Analog/Digital Converter Properties

| | |
|---|---|
| Interval (sec): | 0.200 |
| Rate (pts/sec): | 5 |
| Polarity: | Bipolar |

Channel Summary

| | Name: | Interpreted Value | | |
|---|---|---|---|---|
| No. | Voltage (volts): | Status: | Full Scale: | Interpreted Unit: |
| 1 | | 100.0 | % | 1.0 Used |

### Quantitation Information:

| | |
|---|---|
| Sample Type: | Unknown |
| Dilution Factor: | 1.000000 |
| Custom Data: | |
| Quantitation Table: | |

### Period 1:

| | |
|---|---|
| Scans in Period: | 1495 |
| Relative Start Time: | 0.00 msec |
| Experiments in Period: | 1 |

### Period 1 Experiment 1:

| | |
|---|---|
| Scan Type: | Q1 MS (Q1) |
| Polarity: | Positive |
| Scan Mode: | Profile |
| Resolution Q1: | UNIT |
| Intensity Thres.: | 0.00 cps |
| Settling Time: | 0.0000 ms |
| MR Pause: | 5.0070 ms |
| MCA: | No |
| Center/Width: | No |
| Step Size: | 0.10 amu |

| Start (amu) | Stop (amu) | Time (sec) | Param | Start | Stop |
|---|---|---|---|---|---|
| 50.00 | 1700.00 | 1.60 | CEP | 6.47 | 66.65 |

### Parameter Table (Period 1 Experiment 1)

| | |
|---|---|
| CUR: | 20.00 |
| TEM: | 400.00 |
| GS1: | 20.00 |
| GS2: | 50.00 |
| ihe: | ON |
| IS: | 4500.00 |
| DP | 90.00 |
| EP | 10.00 |

### Example 3

### Determination of the Bio-Therapeutic Potential of Terminalia arjuna Extracts

### A. Antioxidant Assay

The antioxidant activities of natural components may have reciprocal correlation with their reducing potentials. Several methods have been developed to measure the efficacy of dietary antioxidants as pure compounds or in food extracts. These methods focus on different mechanisms of the oxidant defense system i.e. scavenging active oxygen species and hydroxyl radicals, reduction of lipid peroxyl radicals, inhibition of lipid per-oxidation, or chelation of metal ions. In most of the cases irrespective of the stage in the non-enzymatic anti-oxidative activity (scavenging of free radicals, inhibition of lipid per-oxidation, etc.) is mediated by redox reactions.

### 1. DPPH Scavenging Effect

a. Assay Principle

This method is based on the reduction of DPPH, a stable free radical. Due to the odd electron of DPPH, it gives a strong absorption maximum at 517 nm by visible spectroscopy (purple color). As the odd electron of the radical becomes paired off in the presence of hydrogen donor, that is, a free-radical scavenging antioxidant, the absorption strength is decreased, and the resulting de-coloration is stoichiometric with respect to the number of electrons captured. This reaction has widely been used to evaluate the anti-oxidative activity of food and plant extracts.

b. Assay Method

Reactions were performed in 1.25 ml of methanol containing 0.5 mM freshly made DPPH and various amounts of the extract. Reaction mixtures were incubated at 37° C. for 30 min, and the absorbance at 517 nm was measured. This assay was done in triplicate.

Oxidant (DPPH) inhibitory activity (%)={(A₅₁₇Control−A₅₁₇ Sample)/A₅₁₇Control}×100

c. Results and Discussions:

It was found that the reduction of DPPH radical was dose dependent. IC₅₀ is defined as the amount of extract required for 50% inhibition in the levels of free radical. Table 25 gives the IC₅₀ values of Terminalia arjuna bark and fruit extracts.

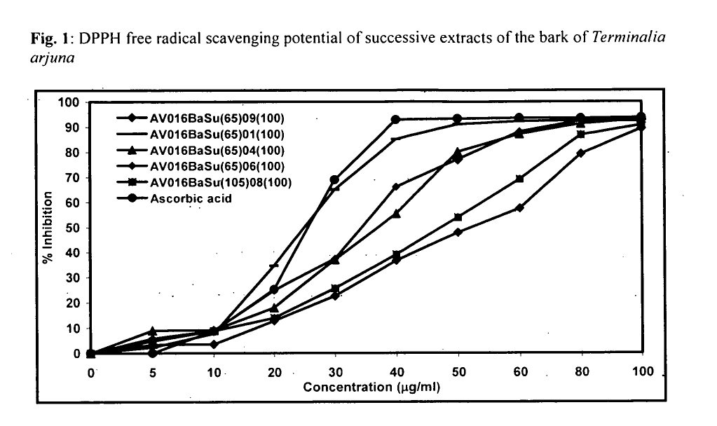

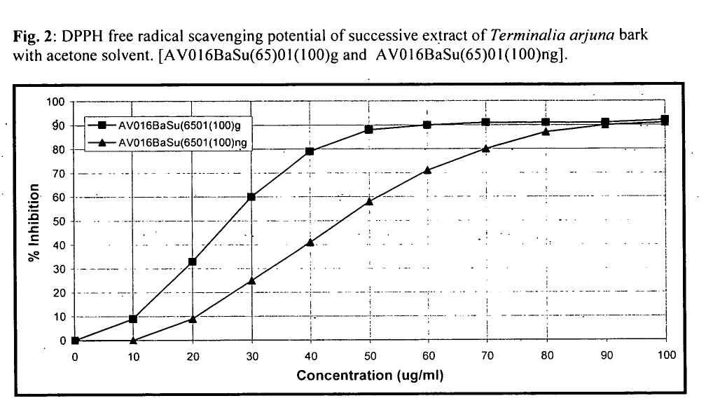

IC₅₀ of Terminalia arjuna successive extracts AV016BaSu(65)01(100), AV016BaSu(65)09(100), AV016BaSu(65)04(100), AV016BaSu(65)06(100), AV016BaSu(105)08(100), AV016BaSu(65)01 (100)g and AV016BaSu(65)01(100)ng was determined as 25.0 μg/ml, 52.8 μg/ml, 36.8 μg/ml, 34.3 μg/ml, 46.4 μg/ml, 26 μg/ml and 46 μg/ml respectively (FIGS. 1 and 2).

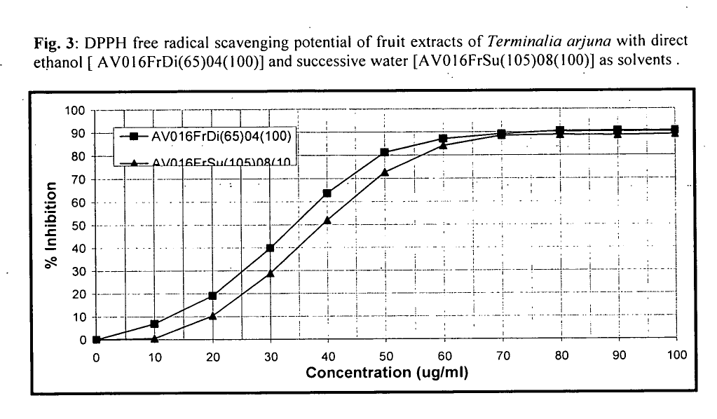

In case of T. arjuna fruit ethanol AV016FrDi(65)04(100) extract and water extract AV016FrSu(105)08(100) was found to be 34 μg/ml and 39 μg/ml respectively (FIG. 3).

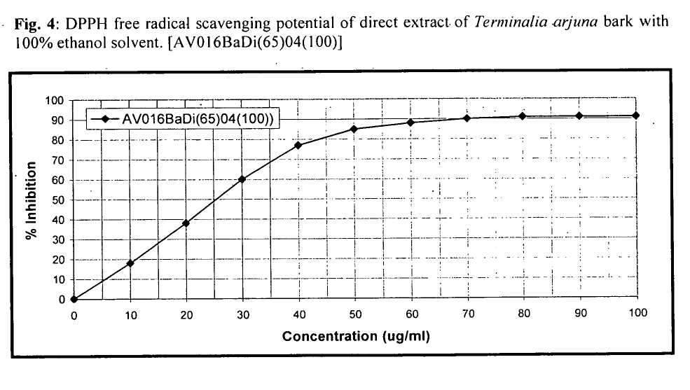

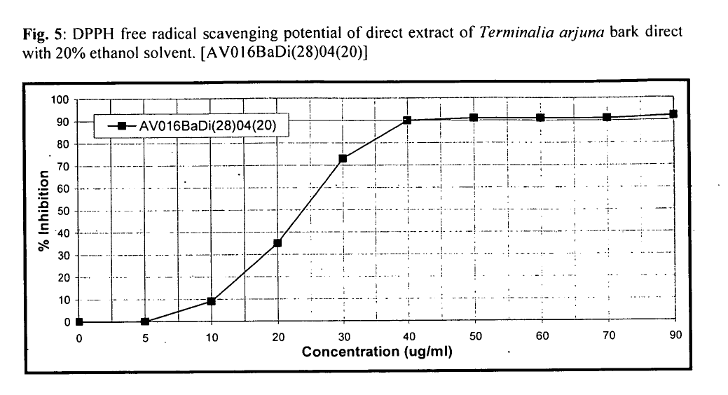

Inhibitory concentration (IC₅₀ values) of Terminalia arjuna direct bark 100% ethanol extract AV016BaDi(65)04(100) was found to be 26 μg/ml whereas that of 20% direct ethanol extract AV016BaDi(28)04(20) was found to be 24 μg/ml (FIGS. 4 and 5).

### CONCLUSIONS

It was seen that IC₅₀ of AV016BaSu(65)01(100), AV016BaSu(65)01(100)g, AV016BaDi(65)04(100) and AV016BaDi(28)04(20) extracts was found to be less than that of ascorbic acid, thereby showing potential anti-oxidation potential.

### B. Antibacterial Assay

### Cultures Tested:

Testing of anti-microbial potential was done against following bacterial strains (Gram negative: Escherichia coli ATCC-10536, Pseudomonas aeruginosa ATCC-9027, Klebsiella pneumoniea ATCC-10031, Bordetella bronchiseptica ATCC-4617; Gram Positive: Staphylococcus aureus ATCC-29737, Streptococcus fecalis ATCC-8043, Micrococcus luteus ATCC-9341, Bacillus subtilis ATCC-6633, Bacillus cereus ATCC-11778, Bacillus pumilus ATCC-14884, Staphylococcus epidermidis ATCC-6358) were selected from the microorganisms given in United states Pharmacopoeia (2000), British Pharmacopoeia (1993) and Indian Pharmacopoeia (1996) for anti-microbial assays.

### Agar Streak Method:

A stock of 100 mg/ml of Ethyl acetate, Acetone, Ethanol, Methanol and Water successive extract from Terminalia arjuna bark and direct ethanol and successive water extracts from Terminalia arjuna fruit was dissolved in DMSO. To determine the antibacterial potential extracts at a concentration of 5 mg/ml and 1 mg/ml were added to 30 ml of luke warm Luria Bertaini agar medium. After the medium was solidified, overnight grown 11 bacterial strains mentioned were taken in loop and streaked on the medium. The plates were incubated at 37° C. for 24 hrs after which the bacterial growth was monitored. Suitable controls were maintained with the extracts and the microorganisms. Luria Bertaini agar medium with and without 1.5% DMSO were used as negative control set, Ciprofloxacin (2 μg/ml) served as positive control.

### Results and Discussion:

Table 2 and 3 enumerates the antibacterial properties of Terminalia arjuna plant part extracts against the standard ATCC bacterial stains used for testing the anti-bacterial potential of the test compounds.

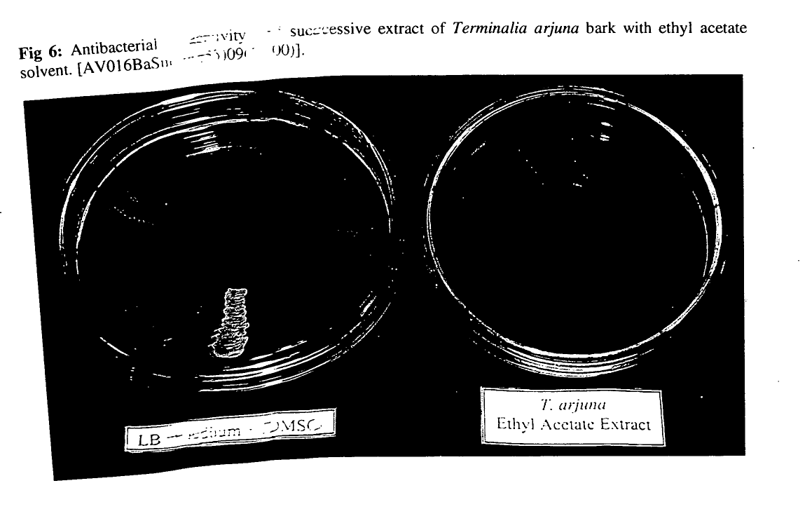

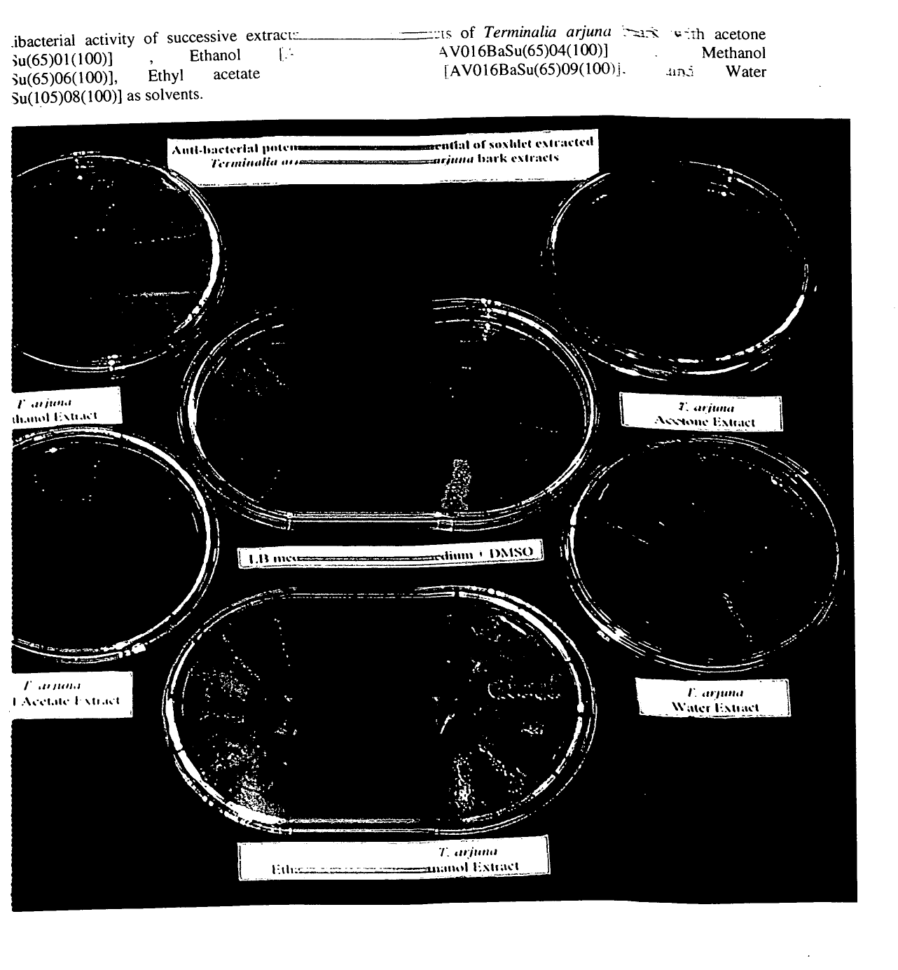

It is observed that at concentration of 5 mg/ml AV016BaSu(65)09(100) extract exhibited a broad antibacterial inhibiting growth of 9 of the 11 bacterial strains tested (FIG. 6). The extract was found to be very effective against the gram-positive bacteria showing inhibition of all the seven gram positive strains tested. Whereas AV016BaSu(65)01 (100), AV016BaSu(65)04(100), AV016BaSu(65)06(100) and AV016BaSu(105)08(100) extract showed inhibition against B. bronchiseptica, S. aureus, S. fecalis and M. luteus (FIG. 7).

At concentration of 1 mg/ml AV016BaSu(65)09(100) extract showed antibacterial activity against B. bronchiseptica, S. aureus and S. fecalis. AV016BaSu(65)01(100) extract showed complete inhibition of growth of S. aureus and S. fecalis whereas showed partial growth inhibition against B. bronchiseptica. AV016BaSu(65)04(100) AV016BaSu(65)06(100) and AV016BaSu(105)08(100) extract showed inhibition against only S. aureus.

Terminalia arjuna direct ethanol fruit extract AV016FrDi(65)04(100) also showed wide spectrum anti-bacterial activity. AV016FrDi(65)04(100) extract at concentration of 5 mg/ml showed bacteriostatic effect against the test strains Escherichia coli, Pseudomonas aeruginosa, Klebsiella pneumoniea, Staphylococcus aureus, Streptococcus fecalis and Micrococcus luteus. AV016FrDi(65)04(100) extract showed completed inhibition of the test stains Bordetella bronchiseptica, Bacillus cereus, Bacillus pumilus and Staphylococcus epidermidis at concentration of 5 mg/ml. At concentration of 1 mg/ml AV016FrDi(65)04(100) extract showed completed inhibition of Bordetella bronchiseptica.

AV016FrSu(105)08(100) extract at concentration of 5 mg/ml showed inhibitory effect only against Bordetella bronchiseptica.

TABLE 1
HPLC fingerprint of extract AV016BaDi(65)04(100)

| | Retention | | | |
|---|---|---|---|---|
| | Time | Area | % Area | Height |
| 1 | 1.746 | 83501 | 0.03 | 18746 |
| 2 | 1.887 | 21939706 | 9.17 | 3508953 |
| 3 | 2.482 | 424664 | 0.18 | 27549 |
| 4 | 3.165 | 694426 | 0.29 | 38196 |
| 5 | 4.499 | 535430 | 0.22 | 20883 |
| 6 | 5.860 | 1140766 | 0.48 | 31447 |
| 7 | 6.488 | 2287279 | 0.96 | 105829 |
| 8 | 7.680 | 2247351 | 0.94 | 73739 |
| 9 | 8.259 | 1172094 | 0.49 | 53148 |
| 10 | 8.702 | 2209397 | 0.92 | 93831 |
| 11 | 9.425 | 2269048 | 0.95 | 89553 |
| 12 | 10.177 | 3848206 | 1.61 | 111334 |
| 13 | 10.485 | 1536103 | 0.64 | 99241 |
| 14 | 10.730 | 1306318 | 0.55 | 102448 |
| 15 | 11.155 | 3705535 | 1.55 | 129030 |
| 16 | 12.263 | 10146875 | 4.24 | 464227 |
| 17 | 12.335 | 6747979 | 2.82 | 548102 |
| 18 | 13.000 | 30474340 | 12.74 | 1778085 |
| 19 | 13.697 | 3437393 | 1.44 | 233361 |
| 20 | 14.232 | 8223596 | 3.44 | 277375 |
| 21 | 14.532 | 14638851 | 6.12 | 819721 |
| 22 | 15.023 | 12657916 | 5.29 | 590365 |
| 23 | 15.452 | 5471266 | 2.29 | 306520 |
| 24 | 15.998 | 16599596 | 6.94 | 1078704 |
| 25 | 16.299 | 4397452 | 1.84 | 318952 |
| 26 | 16.463 | 2424066 | 1.01 | 310668 |
| 27 | 16.863 | 6414264 | 2.68 | 310396 |
| 28 | 17.166 | 12652492 | 5.29 | 355032 |
| 29 | 17.683 | 4940680 | 2.07 | 294832 |
| 30 | 18.118 | 11457746 | 4.79 | 318670 |
| 31 | 18.673 | 2951869 | 1.23 | 232444 |
| 32 | 18.872 | 6643592 | 2.78 | 219376 |
| 33 | 19.475 | 1933618 | 0.81 | 176183 |
| 34 | 19.668 | 2454443 | 1.03 | 182659 |
| 35 | 19.884 | 4858876 | 2.03 | 167366 |
| 36 | 20.329 | 950214 | 0.40 | 139162 |
| 37 | 20.543 | 6254360 | 2.61 | 134329 |
| 38 | 21.427 | 1804592 | 0.75 | 107259 |
| 39 | 21.716 | 7198552 | 3.01 | 109172 |
| 40 | 23.185 | 2885830 | 1.21 | 72715 |
| 41 | 23.905 | 3042057 | 1.27 | 54167 |
| 42 | 24.977 | 665797 | 0.28 | 38055 |
| 43 | 25.258 | 1486354 | 0.62 | 35170 |

TABLE 2
HPLC fingerprint of extract AV016BaDi(28)04(20).

| | Retention | | | |
|---|---|---|---|---|
| | Time | Area | % Area | Height |
| 1 | 1.724 | 259750 | 0.10 | 58489 |
| 2 | 1.897 | 27826788 | 10.53 | 3799266 |
| 3 | 2.486 | 571316 | 0.22 | 50306 |
| 4 | 3.045 | 1106233 | 0.42 | 51910 |
| 5 | 3.732 | 674291 | 0.26 | 29021 |
| 6 | 4.182 | 1056425 | 0.40 | 32454 |
| 7 | 5.572 | 2403677 | 0.91 | 47788 |
| 8 | 6.082 | 3557804 | 1.35 | 161895 |
| 9 | 6.484 | 757991 | 0.29 | 55308 |
| 10 | 7.264 | 3549212 | 1.34 | 97606 |
| 11 | 7.883 | 2512425 | 0.95 | 92337 |
| 12 | 8.299 | 3676625 | 1.39 | 140954 |
| 13 | 9.060 | 4300244 | 1.63 | 140449 |
| 14 | 9.699 | 6186918 | 2.34 | 155916 |
| 15 | 10.124 | 2085208 | 0.79 | 151077 |
| 16 | 10.796 | 7036369 | 2.66 | 179944 |
| 17 | 11.555 | 17109248 | 6.48 | 350960 |
| 18 | 12.574 | 31568344 | 11.95 | 1286226 |
| 19 | 13.038 | 2562494 | 0.97 | 285400 |
| 20 | 13.307 | 4871494 | 1.84 | 288682 |
| 21 | 14.002 | 26201944 | 9.92 | 838854 |
| 22 | 14.597 | 14156853 | 5.36 | 465017 |
| 23 | 15.150 | 4497591 | 1.70 | 322199 |
| 24 | 15.663 | 16422598 | 6.22 | 962872 |
| 25 | 15.912 | 3863537 | 1.46 | 326789 |
| 26 | 16.103 | 6416104 | 2.43 | 315587 |
| 27 | 16.564 | 4817209 | 1.82 | 305649 |
| 28 | 16.767 | 8560395 | 3.24 | 305526 |
| 29 | 17.221 | 2824887 | 1.07 | 283965 |
| 30 | 17.400 | 2813123 | 1.06 | 282612 |
| 31 | 17.661 | 16467191 | 6.23 | 382391 |
| 32 | 18.625 | 6447644 | 2.44 | 200677 |
| 33 | 19.302 | 3348258 | 1.27 | 168434 |
| 34 | 19.568 | 11717199 | 4.44 | 149595 |
| 35 | 21.405 | 4945619 | 1.87 | 87932 |
| 36 | 22.629 | 4393424 | 1.66 | 69440 |
| 37 | 24.162 | 2305522 | 0.87 | 34152 |
| 38 | 25.963 | 255400 | 0.10 | 9559 |

TABLE 3
HPLC fingerprint of extract AV016BaSu(65)09(100)

| | Retention | | | |
|---|---|---|---|---|
| | Time | Area | % Area | Height |
| 1 | 2.475 | 25395907 | 5.13 | 3334075 |
| 2 | 3.244 | 1688929 | 0.34 | 136464 |
| 3 | 4.656 | 9262251 | 1.87 | 280029 |
| 4 | 5.254 | 1188708 | 0.24 | 48754 |
| 5 | 6.603 | 12296585 | 2.48 | 364675 |
| 6 | 6.899 | 5848912 | 1.18 | 389909 |
| 7 | 7.233 | 1367772 | 0.28 | 130776 |
| 8 | 7.428 | 1332175 | 0.27 | 138611 |
| 9 | 7.896 | 7084034 | 1.43 | 280115 |
| 10 | 8.260 | 6705156 | 1.35 | 380995 |
| 11 | 8.576 | 2924953 | 0.59 | 246136 |
| 12 | 8.782 | 2453519 | 0.50 | 228061 |
| 13 | 9.184 | 8669542 | 1.75 | 320819 |
| 14 | 9.521 | 4295969 | 0.87 | 304404 |
| 15 | 9.816 | 4279516 | 0.86 | 324956 |
| 16 | 10.243 | 30792046 | 6.22 | 3248747 |
| 17 | 10.516 | 84937402 | 17.16 | 4333685 |
| 18 | 11.442 | 12774541 | 2.58 | 1056539 |
| 19 | 11.603 | 33723533 | 6.81 | 4613519 |
| 20 | 11.705 | 63394649 | 12.81 | 4142892 |
| 21 | 12.316 | 44870739 | 9.06 | 3991785 |
| 22 | 12.575 | 5106719 | 1.03 | 582499 |
| 23 | 12.754 | 2812210 | 0.57 | 307827 |
| 24 | 12.983 | 7556632 | 1.53 | 836964 |
| 25 | 13.270 | 4609110 | 0.93 | 360754 |
| 26 | 13.418 | 4222758 | 0.85 | 346063 |
| 27 | 13.925 | 16253486 | 3.28 | 1431902 |
| 28 | 14.456 | 7163578 | 1.45 | 605726 |
| 29 | 14.797 | 1631474 | 0.33 | 141283 |
| 30 | 15.240 | 3874910 | 0.78 | 285386 |
| 31 | 15.388 | 3029615 | 0.61 | 225745 |
| 32 | 15.588 | 2719921 | 0.55 | 159504 |
| 33 | 16.139 | 1333368 | 0.27 | 123152 |
| 34 | 16.346 | 3614771 | 0.73 | 345601 |
| 35 | 16.570 | 1244982 | 0.25 | 111424 |
| 36 | 16.813 | 1256674 | 0.25 | 123386 |
| 37 | 16.995 | 1494705 | 0.30 | 110575 |
| 38 | 17.367 | 1278577 | 0.26 | 73925 |
| 39 | 17.823 | 2517030 | 0.51 | 117093 |
| 40 | 18.123 | 2322326 | 0.47 | 179787 |
| 41 | 18.419 | 841112 | 0.17 | 86329 |
| 42 | 18.673 | 2150256 | 0.43 | 93219 |
| 43 | 18.954 | 429553 | 0.09 | 73333 |
| 44 | 19.255 | 1852803 | 0.37 | 78786 |
| 45 | 19.627 | 1385755 | 0.28 | 84299 |
| 46 | 19.912 | 1175927 | 0.24 | 106435 |
| 47 | 20.169 | 2238619 | 0.45 | 198261 |
| 48 | 20.362 | 1903752 | 0.38 | 136402 |
| 49 | 20.725 | 1388092 | 0.28 | 86158 |
| 50 | 21.002 | 2038182 | 0.41 | 112055 |
| 51 | 21.221 | 957990 | 0.19 | 84615 |
| 52 | 21.507 | 1049496 | 0.21 | 79603 |
| 53 | 21.799 | 1772753 | 0.36 | 102185 |
| 54 | 22.203 | 1643957 | 0.33 | 76435 |
| 55 | 22.543 | 831012 | 0.17 | 71872 |
| 56 | 22.690 | 572833 | 0.12 | 72239 |
| 57 | 22.941 | 3535908 | 0.71 | 220985 |
| 58 | 23.299 | 2133293 | 0.43 | 142496 |
| 59 | 23.648 | 2144927 | 0.43 | 151460 |
| 60 | 23.960 | 1493722 | 0.30 | 100416 |
| 61 | 24.429 | 2297448 | 0.46 | 123572 |
| 62 | 24.639 | 908084 | 0.18 | 85150 |
| 63 | 24.933 | 2402551 | 0.49 | 104466 |
| 64 | 25.280 | 583388 | 0.12 | 83878 |
| 65 | 25.564 | 1925541 | 0.39 | 92355 |
| 66 | 25.800 | 1153719 | 0.23 | 90624 |
| 67 | 25.974 | 1727846 | 0.35 | 88759 |
| 68 | 26.404 | 1397501 | 0.28 | 91036 |
| 69 | 26.591 | 795071 | 0.16 | 89416 |
| 70 | 26.751 | 1482526 | 0.30 | 89528 |
| 71 | 27.018 | 763878 | 0.15 | 86118 |
| 72 | 27.157 | 1700407 | 0.34 | 86915 |
| 73 | 27.647 | 2637337 | 0.53 | 78919 |
| 74 | 28.193 | 4360949 | 0.88 | 662358 |

TABLE 4
HPLC fingerprint of extract AV016BaSu(65)01(100).

| | Retention | | | |
|---|---|---|---|---|
| | Time | Area | % Area | Height |
| 1 | 2.391 | 6928 | 0.00 | 852 |
| 2 | 2.556 | 1739539 | 0.82 | 304280 |
| 3 | 3.313 | 84646 | 0.04 | 9680 |
| 4 | 3.686 | 223139 | 0.10 | 9176 |
| 5 | 4.269 | 63325 | 0.03 | 3419 |
| 6 | 5.024 | 13893 | 0.01 | 769 |
| 7 | 6.157 | 4281 | 0.00 | 572 |
| 8 | 6.736 | 197041 | 0.09 | 6916 |
| 9 | 7.391 | 117641 | 0.06 | 5898 |
| 10 | 7.878 | 616604 | 0.29 | 17813 |
| 11 | 8.308 | 278599 | 0.13 | 15829 |
| 12 | 8.860 | 611104 | 0.29 | 22047 |
| 13 | 9.391 | 187896 | 0.09 | 12237 |
| 14 | 9.564 | 276548 | 0.13 | 14167 |
| 15 | 12.058 | 8846367 | 4.16 | 183309 |
| 16 | 12.644 | 751247 | 0.35 | 38688 |
| 17 | 13.199 | 1719360 | 0.81 | 43466 |
| 18 | 13.988 | 1735451 | 0.82 | 56406 |
| 19 | 14.555 | 2169673 | 1.02 | 76948 |
| 20 | 14.875 | 2254289 | 1.06 | 115963 |
| 21 | 15.626 | 4155110 | 1.95 | 123796 |
| 22 | 15.956 | 1080279 | 0.51 | 86186 |
| 23 | 16.580 | 5912054 | 2.78 | 175797 |
| 24 | 17.766 | 7200465 | 3.38 | 141141 |
| 25 | 18.423 | 2986827 | 1.40 | 111890 |
| 26 | 19.067 | 6971841 | 3.28 | 151382 |
| 27 | 19.737 | 4017460 | 1.89 | 145309 |
| 28 | 20.104 | 1488984 | 0.70 | 136621 |
| 29 | 20.459 | 4173594 | 1.96 | 175505 |
| 30 | 20.795 | 3235535 | 1.52 | 167773 |
| 31 | 21.079 | 1788929 | 0.84 | 164753 |
| 32 | 21.272 | 4796914 | 2.25 | 175363 |
| 33 | 21.910 | 4124406 | 1.94 | 168234 |
| 34 | 22.267 | 6071222 | 2.85 | 185861 |
| 35 | 22.997 | 5930428 | 2.79 | 190259 |
| 36 | 23.451 | 5868309 | 2.76 | 231002 |
| 37 | 23.866 | 16287689 | 7.65 | 1076501 |
| 38 | 24.287 | 4035419 | 1.90 | 266032 |
| 39 | 24.627 | 3569391 | 1.68 | 266672 |
| 40 | 24.844 | 42015183 | 19.75 | 2046204 |
| 41 | 26.107 | 8757964 | 4.12 | 239370 |
| 42 | 26.900 | 25586681 | 12.02 | 821474 |
| 43 | 27.926 | 7429374 | 3.49 | 323025 |
| 44 | 28.156 | 13400123 | 6.30 | 654328 |

TABLE 5
HPLC fingerprint of extract AV016BaSu(65)01(100)ng.

| | Retention | | | |
|---|---|---|---|---|
| | Time | Area | % Area | Height |
| 1 | 2.579 | 1411822 | 0.59 | 207187 |
| 2 | 3.336 | 80911 | 0.03 | 9988 |
| 3 | 3.518 | 367100 | 0.15 | 14428 |
| 4 | 4.289 | 54629 | 0.02 | 3130 |
| 5 | 4.766 | 46285 | 0.02 | 1682 |
| 6 | 6.659 | 58963 | 0.02 | 2489 |
| 7 | 7.316 | 3635 | 0.00 | 454 |
| 8 | 7.852 | 277194 | 0.12 | 10198 |
| 9 | 8.240 | 150308 | 0.06 | 8971 |
| 10 | 8.774 | 321493 | 0.14 | 13773 |
| 11 | 9.207 | 113505 | 0.05 | 7075 |
| 12 | 9.458 | 151700 | 0.06 | 8682 |
| 13 | 10.403 | 841535 | 0.35 | 23389 |
| 14 | 11.877 | 5609438 | 2.36 | 151067 |
| 15 | 12.469 | 548895 | 0.23 | 28503 |
| 16 | 12.997 | 1315579 | 0.55 | 34524 |
| 17 | 13.798 | 1396765 | 0.59 | 46557 |
| 18 | 14.301 | 1552115 | 0.65 | 56533 |
| 19 | 14.658 | 1693118 | 0.71 | 88234 |
| 20 | 15.378 | 2309177 | 0.97 | 76351 |
| 21 | 15.719 | 1849902 | 0.78 | 89567 |
| 22 | 16.341 | 3751422 | 1.58 | 131368 |
| 23 | 16.651 | 705622 | 0.30 | 59259 |
| 24 | 17.537 | 5824011 | 2.45 | 121156 |
| 25 | 18.170 | 2202916 | 0.93 | 92276 |
| 26 | 19.153 | 16377423 | 6.89 | 384476 |
| 27 | 20.514 | 11154160 | 4.69 | 206032 |
| 28 | 21.248 | 27468304 | 11.56 | 1141614 |
| 29 | 22.005 | 3503203 | 1.47 | 178231 |
| 30 | 22.665 | 5722669 | 2.41 | 184191 |
| 31 | 23.185 | 15337448 | 6.45 | 535651 |
| 32 | 23.666 | 13965998 | 5.88 | 926816 |
| 33 | 24.155 | 2815614 | 1.18 | 263179 |
| 34 | 24.360 | 4975496 | 2.09 | 287271 |
| 35 | 24.677 | 30074237 | 12.65 | 1853497 |
| 36 | 25.316 | 9050757 | 3.81 | 411682 |
| 37 | 25.712 | 6189308 | 2.60 | 264816 |
| 38 | 26.607 | 20990123 | 8.83 | 826640 |
| 39 | 26.996 | 4842260 | 2.04 | 310728 |
| 40 | 27.520 | 16829287 | 7.08 | 814850 |
| 41 | 27.942 | 15732031 | 6.62 | 542909 |

TABLE 6
HPLC fingerprint of extract AV016BaSu(65)01(100)g.

| | Retention | | | |
|---|---|---|---|---|
| | Time | Area | % Area | Height |
| 1 | 2.384 | 12028 | 0.01 | 947 |
| 2 | 2.570 | 2633549 | 1.23 | 435477 |
| 3 | 3.507 | 735108 | 0.34 | 33546 |
| 4 | 5.043 | 9805 | 0.00 | 651 |
| 5 | 6.822 | 78175 | 0.04 | 3779 |
| 6 | 7.045 | 83478 | 0.04 | 5089 |
| 7 | 7.203 | 66115 | 0.03 | 5355 |
| 8 | 7.820 | 581086 | 0.27 | 16595 |
| 9 | 8.749 | 775713 | 0.36 | 25475 |
| 10 | 9.447 | 325440 | 0.15 | 11555 |
| 11 | 10.387 | 882547 | 0.41 | 20400 |
| 12 | 11.017 | 953840 | 0.45 | 28086 |
| 13 | 11.853 | 2915239 | 1.36 | 86837 |
| 14 | 12.467 | 565867 | 0.26 | 27905 |
| 15 | 13.088 | 924183 | 0.43 | 31678 |
| 16 | 13.160 | 468207 | 0.22 | 31979 |
| 17 | 13.766 | 1224906 | 0.57 | 43299 |
| 18 | 14.633 | 3503010 | 1.64 | 84712 |
| 19 | 15.471 | 1966123 | 0.92 | 60085 |
| 20 | 15.728 | 1016548 | 0.48 | 66817 |
| 21 | 16.317 | 3374916 | 1.58 | 110591 |
| 22 | 16.627 | 663866 | 0.31 | 60558 |
| 23 | 17.526 | 4303710 | 2.01 | 88642 |
| 24 | 17.688 | 741644 | 0.35 | 86630 |
| 25 | 18.026 | 1554964 | 0.73 | 90925 |
| 26 | 18.356 | 1800352 | 0.84 | 101584 |
| 27 | 18.852 | 3360093 | 1.57 | 117653 |
| 28 | 19.607 | 6271811 | 2.93 | 186866 |
| 29 | 19.840 | 4324472 | 2.02 | 201700 |
| 30 | 20.208 | 6132533 | 2.87 | 247918 |
| 31 | 20.820 | 2351992 | 1.10 | 151443 |
| 32 | 21.075 | 3520296 | 1.65 | 173642 |
| 33 | 23.221 | 22630392 | 10.58 | 235431 |
| 34 | 23.623 | 17865849 | 8.35 | 1114913 |
| 35 | 24.057 | 4708441 | 2.20 | 290733 |
| 36 | 24.359 | 4086311 | 1.91 | 276895 |
| 37 | 24.661 | 35429569 | 16.57 | 1765464 |
| 38 | 26.639 | 30653977 | 14.33 | 480095 |
| 39 | 26.977 | 5710437 | 2.67 | 393204 |
| 40 | 27.595 | 12852254 | 6.01 | 398867 |
| 41 | 27.911 | 21792801 | 10.19 | 563202 |

TABLE 7
HPLC fingerprint of extract AV016BaSu(65)04(100).

| | Retention | | | |
|---|---|---|---|---|
| | Time | Area | % Area | Height |
| 1 | 1.241 | 10089 | 0.00 | 743 |
| 2 | 1.737 | 139563 | 0.04 | 29368 |
| 3 | 1.897 | 10508564 | 3.02 | 2120419 |
| 4 | 2.474 | 168462 | 0.05 | 20793 |
| 5 | 3.560 | 244245 | 0.07 | 13079 |
| 6 | 5.156 | 118208 | 0.03 | 5005 |
| 7 | 6.455 | 515048 | 0.15 | 12630 |
| 8 | 7.011 | 397795 | 0.11 | 16720 |
| 9 | 7.792 | 1203998 | 0.35 | 35331 |
| 10 | 8.408 | 1782066 | 0.51 | 81114 |
| 11 | 9.287 | 3153097 | 0.90 | 85209 |
| 12 | 10.010 | 3363741 | 0.97 | 117298 |
| 13 | 10.521 | 3211695 | 0.92 | 129563 |
| 14 | 10.919 | 3272829 | 0.94 | 151739 |
| 15 | 11.201 | 2910895 | 0.84 | 187760 |
| 16 | 11.469 | 2011099 | 0.58 | 187543 |
| 17 | 12.570 | 22668982 | 6.51 | 608697 |
| 18 | 13.072 | 22315645 | 6.40 | 1372355 |
| 19 | 13.943 | 15908037 | 4.56 | 466306 |
| 20 | 14.267 | 13002097 | 3.73 | 755966 |
| 21 | 14.499 | 14043370 | 4.03 | 677681 |
| 22 | 15.168 | 18500419 | 5.31 | 803270 |
| 23 | 15.443 | 8340109 | 2.39 | 561189 |
| 24 | 15.645 | 7732494 | 2.22 | 554586 |
| 25 | 15.999 | 15671975 | 4.50 | 569336 |
| 26 | 16.333 | 14724201 | 4.23 | 556235 |
| 27 | 16.825 | 11690960 | 3.35 | 545534 |
| 28 | 17.178 | 11285355 | 3.24 | 522522 |
| 29 | 18.532 | 79310513 | 22.76 | 612406 |
| 30 | 19.864 | 57401105 | 16.47 | 441406 |
| 31 | 25.389 | 2531990 | 0.73 | 47750 |
| 32 | 29.165 | 4724 | 0.00 | 733 |
| 33 | 29.850 | 337961 | 0.10 | 60806 |

TABLE 8
HPLC fingerprint of extract AV016BaSu(65)06(100).

| | Retention | | | |
|---|---|---|---|---|
| | Time | Area | % Area | Height |
| 1 | 1.726 | 325346 | 0.17 | 47632 |
| 2 | 1.893 | 23371224 | 12.48 | 3640871 |
| 3 | 2.482 | 714853 | 0.38 | 60900 |
| 4 | 3.177 | 1875237 | 1.00 | 99118 |
| 5 | 3.690 | 485133 | 0.26 | 25416 |
| 6 | 4.103 | 679406 | 0.36 | 27133 |
| 7 | 4.359 | 352324 | 0.19 | 24825 |
| 8 | 4.857 | 272252 | 0.15 | 14769 |
| 9 | 5.596 | 1486044 | 0.79 | 49063 |
| 10 | 6.072 | 4857288 | 2.59 | 165611 |
| 11 | 7.074 | 715830 | 0.38 | 46771 |
| 12 | 7.247 | 928001 | 0.50 | 52209 |
| 13 | 7.925 | 1065970 | 0.57 | 45480 |
| 14 | 8.330 | 2132695 | 1.14 | 79394 |
| 15 | 9.096 | 2308356 | 1.23 | 80800 |
| 16 | 9.743 | 2396105 | 1.28 | 79390 |
| 17 | 11.179 | 12808928 | 6.84 | 205854 |
| 18 | 12.413 | 25125074 | 13.42 | 602504 |
| 19 | 13.130 | 1899754 | 1.01 | 160549 |
| 20 | 14.031 | 28439970 | 15.19 | 935325 |
| 21 | 14.646 | 9859006 | 5.27 | 326112 |
| 22 | 15.208 | 3351792 | 1.79 | 211034 |
| 23 | 15.744 | 11230538 | 6.00 | 540161 |
| 24 | 16.030 | 4547672 | 2.43 | 212887 |
| 25 | 16.651 | 5194350 | 2.77 | 232212 |
| 26 | 16.896 | 3162049 | 1.69 | 216298 |
| 27 | 17.244 | 5839204 | 3.12 | 208158 |
| 28 | 17.782 | 12488631 | 6.67 | 281615 |
| 29 | 18.729 | 4904610 | 2.62 | 146569 |
| 30 | 19.386 | 2196346 | 1.17 | 115299 |
| 31 | 19.673 | 8273125 | 4.42 | 99688 |
| 32 | 21.529 | 667601 | 0.36 | 50722 |
| 33 | 21.763 | 1946885 | 1.04 | 46660 |
| 34 | 22.802 | 1281629 | 0.68 | 26705 |
| 35 | 26.305 | 16760 | 0.01 | 1806 |
| 36 | 27.757 | 52103 | 0.03 | 5397 |

TABLE 9
HPLC fingerprint of extract AV016BaSu(105)08(100).

| | Retention | | | |
|---|---|---|---|---|
| | Time | Area | % Area | Height |
| 1 | 2.531 | 251954 | 2.83 | 45170 |
| 2 | 3.153 | 91508 | 1.03 | 7618 |
| 3 | 3.590 | 53827 | 0.60 | 2846 |
| 4 | 5.809 | 95383 | 1.07 | 5176 |
| 5 | 6.237 | 84290 | 0.95 | 3408 |
| 6 | 7.377 | 237161 | 2.66 | 7025 |
| 7 | 7.781 | 208394 | 2.34 | 12462 |
| 8 | 7.968 | 105188 | 1.18 | 10471 |
| 9 | 8.523 | 386758 | 4.34 | 15438 |
| 10 | 8.922 | 255053 | 2.86 | 13182 |
| 11 | 9.164 | 103465 | 1.16 | 11779 |
| 12 | 9.356 | 146409 | 1.64 | 11519 |
| 13 | 9.690 | 263555 | 2.96 | 11664 |
| 14 | 10.035 | 197458 | 2.22 | 12951 |
| 15 | 10.220 | 188840 | 2.12 | 14479 |
| 16 | 10.471 | 188182 | 2.11 | 14258 |
| 17 | 10.814 | 371997 | 4.18 | 13832 |
| 18 | 11.142 | 112966 | 1.27 | 12605 |
| 19 | 11.344 | 318863 | 3.58 | 13928 |
| 20 | 11.735 | 280148 | 3.15 | 11849 |
| 21 | 12.451 | 609274 | 6.84 | 21533 |
| 22 | 13.107 | 585271 | 6.57 | 38319 |
| 23 | 13.648 | 210651 | 2.37 | 8967 |
| 24 | 14.205 | 227248 | 2.55 | 13908 |
| 25 | 14.513 | 2594830 | 29.14 | 208042 |
| 26 | 15.951 | 40742 | 0.46 | 3155 |
| 27 | 16.683 | 52708 | 0.59 | 5023 |
| 28 | 17.691 | 70180 | 0.79 | 6181 |
| 29 | 24.062 | 198328 | 2.23 | 4386 |
| 30 | 24.791 | 13467 | 0.15 | 1398 |
| 31 | 24.993 | 12465 | 0.14 | 996 |
| 32 | 25.557 | 2542 | 0.03 | 494 |
| 33 | 26.037 | 62889 | 0.71 | 3648 |
| 34 | 26.578 | 50279 | 0.56 | 5059 |
| 35 | 28.119 | 5587 | 0.06 | 665 |
| 36 | 28.951 | 42494 | 0.48 | 3689 |
| 37 | 29.707 | 184276 | 2.07 | 17288 |

TABLE 10
HPLC fingerprint of extract AV016Fr(105)08(100).

| | Retention | | | |
|---|---|---|---|---|
| | Time | Area | % Area | Height |
| 1 | 2.238 | 10762330 | 4.73 | 1150702 |
| 2 | 2.553 | 5628824 | 2.47 | 655829 |
| 3 | 2.914 | 2203056 | 0.97 | 125091 |
| 4 | 3.208 | 3512641 | 1.54 | 318280 |
| 5 | 3.357 | 1070868 | 0.47 | 212573 |
| 6 | 3.602 | 1899749 | 0.84 | 121669 |
| 7 | 4.052 | 3407226 | 1.50 | 136020 |
| 8 | 4.503 | 2919013 | 1.28 | 94633 |
| 9 | 5.093 | 2190949 | 0.96 | 89619 |
| 10 | 5.889 | 12371237 | 5.44 | 338787 |
| 11 | 6.223 | 10361831 | 4.55 | 315486 |
| 12 | 7.163 | 4782640 | 2.10 | 237340 |
| 13 | 7.700 | 40604091 | 17.85 | 1251589 |
| 14 | 9.163 | 11851832 | 5.21 | 268920 |
| 15 | 9.857 | 3502688 | 1.54 | 228636 |
| 16 | 10.125 | 4862586 | 2.14 | 227948 |
| 17 | 10.448 | 2710010 | 1.19 | 231182 |
| 18 | 10.625 | 2889073 | 1.27 | 233695 |
| 19 | 10.818 | 1656694 | 0.73 | 207765 |
| 20 | 11.218 | 23908811 | 10.51 | 1250520 |
| 21 | 12.067 | 40804495 | 17.94 | 1657258 |
| 22 | 13.556 | 507546 | 0.22 | 127495 |
| 23 | 13.790 | 6006453 | 2.64 | 134107 |
| 24 | 14.940 | 8534623 | 3.75 | 102587 |
| 25 | 17.771 | 960263 | 0.42 | 27876 |
| 26 | 18.313 | 820905 | 0.36 | 25276 |
| 27 | 20.696 | 13722 | 0.01 | 1129 |
| 28 | 23.988 | 77588 | 0.03 | 2021 |
| 29 | 24.355 | 9612 | 0.00 | 1658 |
| 30 | 25.622 | 204766 | 0.09 | 9215 |
| 31 | 25.787 | 301167 | 0.13 | 11470 |
| 32 | 26.472 | 205806 | 0.09 | 9613 |
| 33 | 27.231 | 486773 | 0.21 | 12154 |
| 34 | 28.270 | 5013669 | 2.20 | 94934 |
| 35 | 29.001 | 2956931 | 1.30 | 213028 |
| 36 | 29.400 | 7494149 | 3.29 | 238402 |

TABLE 11
MS Fingerprint of extract AV016BaDi(28)04(20)
Peak List:

| Time | | | Height | | Width | |
|---|---|---|---|---|---|---|
| (min) | Area (counts) | % Area | (cps) | % Height | (min) | Baseline Type |
| 2.1775 | 1.06E+08 | 12.3131 | 1.97E+07 | 17.7412 | 0.1874 | Valley |
| 2.3167 | 3.07E+08 | 35.522 | 3.45E+07 | 31.1894 | 0.2408 | Valley |
| 2.8588 | 2.07E+07 | 2.398 | 2.52E+06 | 2.2768 | 0.2408 | Base to Base |
| 3.2879 | 1.84E+07 | 2.1314 | 2.99E+06 | 2.7 | 0.1873 | Base to Base |
| 3.7661 | 1.19E+07 | 1.3822 | 1.74E+06 | 1.5668 | 0.2408 | Base to Base |
| 5.0086 | 9.58E+06 | 1.1091 | 1.08E+06 | 0.9759 | 0.2941 | Base to Base |
| 6.1538 | 8.09E+06 | 0.9365 | 1.03E+06 | 0.9294 | 0.2141 | Base to Base |
| 6.8962 | 6.00E+06 | 0.6949 | 1.39E+06 | 1.2572 | 0.1605 | Base to Base |
| 7.7876 | 8.31E+06 | 0.9627 | 1.05E+06 | 0.9452 | 0.2676 | Valley |
| 7.9719 | 1.18E+07 | 1.3624 | 1.89E+06 | 1.7057 | 0.214 | Valley |
| 10.735 | 8.06E+06 | 0.9334 | 1.45E+06 | 1.3135 | 0.2408 | Base to Base |
| 12.0425 | 2.48E+06 | 0.2866 | 6.74E+05 | 0.6081 | 0.107 | Base to Base |
| 12.3092 | 1.54E+06 | 0.1783 | 8.28E+05 | 0.7472 | 0.0803 | Base to Base |
| 12.7311 | 4.83E+06 | 0.5597 | 9.68E+05 | 0.8739 | 0.1605 | Base to Base |
| 14.3893 | 3.76E+06 | 0.4351 | 9.54E+05 | 0.8611 | 0.1605 | Base to Base |
| 15.0886 | 3.66E+06 | 0.4237 | 9.12E+05 | 0.8235 | 0.1338 | Base to Base |
| 17.5521 | 1.82E+07 | 2.1125 | 2.34E+06 | 2.1145 | 0.3211 | Base to Base |
| 17.9476 | 1.90E+07 | 2.1971 | 1.86E+06 | 1.6753 | 0.2408 | Base to Base |
| 19.5586 | 1.44E+07 | 1.6648 | 1.53E+06 | 1.3819 | 0.3211 | Base to Base |
| 20.3886 | 4.78E+06 | 0.553 | 1.22E+06 | 1.1028 | 0.1605 | Base to Base |
| 21.5431 | 1.49E+07 | 1.7195 | 1.31E+06 | 1.1854 | 0.3746 | Base to Base |
| 23.6233 | 8.28E+06 | 0.9586 | 1.77E+06 | 1.5935 | 0.1873 | Base to Base |
| 24.5583 | 8.58E+06 | 0.9939 | 1.18E+06 | 1.0651 | 0.2676 | Base to Base |
| 25.1938 | 9.77E+06 | 1.131 | 1.24E+06 | 1.117 | 0.3211 | Base to Base |
| 25.6005 | 5.11E+07 | 5.9143 | 5.26E+06 | 4.7467 | 0.3478 | Base to Base |
| 25.9137 | 1.34E+07 | 1.5528 | 1.76E+06 | 1.5921 | 0.2141 | Base to Base |
| 26.7469 | 1.82E+07 | 2.1106 | 2.05E+06 | 1.8533 | 0.2943 | Base to Base |
| 27.3985 | 1.09E+07 | 1.2594 | 1.39E+06 | 1.2536 | 0.2675 | Base to Base |
| 27.6349 | 4.60E+06 | 0.5329 | 1.06E+06 | 0.9601 | 0.1338 | Base to Base |
| 28.7272 | 1.89E+07 | 2.1893 | 1.77E+06 | 1.5968 | 0.3478 | Base to Base |
| 28.8934 | 1.03E+07 | 1.1905 | 2.09E+06 | 1.8883 | 0.1338 | Base to Base |
| 29.1613 | 3.01E+06 | 0.3489 | 9.23E+05 | 0.8329 | 0.1338 | Base to Base |
| 29.9922 | 8.20E+07 | 9.495 | 5.03E+06 | 4.5397 | 0.6957 | Base to Base |
| 34.628 | 1.16E+07 | 1.3485 | 1.01E+06 | 0.9137 | 0.3746 | Base to Base |
| 36.8649 | 2.86E+06 | 0.3315 | 8.91E+05 | 0.804 | 0.107 | Base to Base |
| 37.1874 | 1.77E+06 | 0.2051 | 6.81E+05 | 0.6146 | 0.0802 | Base to Base |
| 37.7494 | 4.85E+06 | 0.5616 | 7.24E+05 | 0.6535 | 0.1873 | Base to Base |

TABLE 12
MS Fingerprint of extract AV016BaDi(28)04(20)
Peak List:

| Time | Area | | | | | |
|---|---|---|---|---|---|---|
| (min) | (counts) | % Area | Height (cps) | % Height | Width (min) | Baseline Type |
| 2.1961 | 2.85E+08 | 24.4168 | 3.04E+07 | 25.3851 | 0.2676 | Valley |
| 2.4834 | 4.77E+08 | 40.8241 | 2.58E+07 | 21.5134 | 0.5084 | Valley |
| 3.4214 | 7.47E+06 | 0.6387 | 1.44E+06 | 1.2067 | 0.1873 | Base to Base |
| 4.4188 | 3.03E+06 | 0.2592 | 1.25E+06 | 1.0442 | 0.107 | Base to Base |
| 7.9809 | 8.39E+06 | 0.7181 | 1.33E+06 | 1.1079 | 0.1873 | Base to Base |
| 9.6559 | 5.75E+06 | 0.4914 | 1.14E+06 | 0.9551 | 0.1873 | Base to Base |
| 11.4246 | 2.60E+06 | 0.2223 | 8.53E+06 | 0.7128 | 0.1338 | Base to Base |
| 12.387 | 6.72E+06 | 0.575 | 1.29E+06 | 1.0785 | 0.1873 | Base to Base |
| 12.7886 | 3.28E+07 | 2.8027 | 4.15E+06 | 3.4643 | 0.2408 | Base to Base |
| 14.0996 | 3.48E+06 | 0.2974 | 1.12E+06 | 0.9338 | 0.107 | Base to Base |
| 14.8736 | 3.87E+06 | 0.3307 | 5.13E+05 | 0.4282 | 0.1605 | Base to Base |
| 15.1974 | 2.00E+06 | 0.1714 | 1.08E+06 | 0.9018 | 0.0803 | Base to Base |
| 16.2118 | 1.20E+07 | 1.0275 | 1.28E+06 | 1.0691 | 0.2676 | Base to Base |
| 17.1494 | 6.42E+06 | 0.5495 | 1.26E+06 | 1.0499 | 0.214 | Base to Base |
| 17.3337 | 1.35E+07 | 1.157 | 2.57E+06 | 2.1439 | 0.1873 | Base to Base |
| 17.6906 | 3.73E+07 | 3.1891 | 2.51E+06 | 2.0939 | 0.4013 | Valley |
| 17.8922 | 9.86E+06 | 0.8435 | 1.99E+06 | 1.6607 | 0.1338 | Valley |
| 18.3572 | 4.74E+06 | 0.4051 | 2.29E+06 | 1.9131 | 0.0803 | Base to Base |
| 18.7319 | 9.90E+06 | 0.8468 | 1.37E+06 | 1.144 | 0.2408 | Base to Base |
| 19.6901 | 6.66E+06 | 0.5693 | 1.68E+06 | 1.4049 | 0.1338 | Base to Base |
| 20.1151 | 1.26E+07 | 1.082 | 1.77E+06 | 1.4749 | 0.2408 | Base to Base |
| 21.084 | 5.17E+06 | 0.4419 | 1.45E+06 | 1.2082 | 0.1338 | Base to Base |
| 21.4301 | 6.15E+06 | 0.5263 | 2.11E+06 | 1.7665 | 0.0803 | Valley |
| 21.5887 | 1.05E+07 | 0.8972 | 2.53E+06 | 2.1134 | 0.1605 | Valley |
| 23.7467 | 8.39E+06 | 0.7173 | 1.24E+06 | 1.0376 | 0.2675 | Base to Base |
| 23.9916 | 4.44E+06 | 0.3795 | 8.19E+05 | 0.6845 | 0.1338 | Base to Base |
| 24.7774 | 6.82E+06 | 0.5835 | 1.14E+06 | 0.9516 | 0.2943 | Base to Base |
| 25.5831 | 2.43E+07 | 2.0804 | 2.38E+06 | 1.9909 | 0.3211 | Base to Base |
| 25.9059 | 1.37E+07 | 1.1725 | 2.04E+06 | 1.7081 | 0.2675 | Base to Base |
| 26.2502 | 1.00E+07 | 0.8555 | 1.08E+06 | 0.9016 | 0.2408 | Base to Base |
| 26.7168 | 1.72E+07 | 1.471 | 2.26E+06 | 1.8859 | 0.2408 | Valley |
| 26.9142 | 2.14E+07 | 1.8288 | 2.20E+06 | 1.8395 | 0.2676 | Valley |
| 28.3515 | 3.10E+07 | 2.6494 | 3.47E+06 | 2.8963 | 0.4281 | Valley |
| 28.6747 | 2.53E+07 | 2.1615 | 3.53E+06 | 2.9518 | 0.3478 | Valley |
| 28.9455 | 4.47E+06 | 0.3823 | 1.21E+06 | 1.0119 | 0.1338 | Base to Base |
| 29.6212 | 7.70E+06 | 0.6586 | 7.87E+05 | 0.6574 | 0.2408 | Base to Base |
| 30.0197 | 1.10E+07 | 0.939 | 2.58E+06 | 2.1565 | 0.1873 | Base to Base |
| 30.4766 | 9.79E+06 | 0.8377 | 1.86E+06 | 1.552 | 0.1605 | Base to Base |

TABLE 13
MS Fingerprint of extract AV016BaDi(65)04(100)
Peak List:

| | Area | | | | Width | |
|---|---|---|---|---|---|---|
| Time (min) | (counts) | % Area | Height (cps) | % Height | (min) | Baseline Type |
| 1.6819 | 5.13E+07 | 3.3673 | 1.07E+07 | 7.702 | 0.1873 | Base to Base |
| 2.1223 | 4.05E+07 | 2.6634 | 4.33E+06 | 3.1211 | 0.2408 | Base to Base |
| 2.4137 | 3.19E+08 | 20.9505 | 4.49E+07 | 32.3621 | 0.3479 | Base to Base |
| 35.37 | 7.19E+07 | 4.7236 | 3.26E+06 | 2.3544 | 0.6422 | Base to Base |
| 43.1584 | 2.30E+08 | 15.0758 | 1.02E+07 | 7.3293 | 0.7759 | Base to Base |
| 46.4236 | 1.38E+07 | 0.9069 | 1.66E+06 | 1.1971 | 0.3746 | Base to Base |
| 48.1523 | 2.43E+07 | 1.5953 | 2.55E+06 | 1.8369 | 0.3211 | Base to Base |
| 48.5731 | 7.28E+07 | 4.7831 | 6.02E+06 | 4.3446 | 0.3746 | Base to Base |
| 49.1844 | 4.59E+07 | 3.0145 | 4.22E+06 | 3.0466 | 0.4014 | Base to Base |
| 49.7221 | 1.53E+08 | 10.0401 | 1.05E+07 | 7.5644 | 0.6421 | Base to Base |
| 50.41 | 1.98E+08 | 13.0047 | 1.31E+07 | 9.4266 | 0.6422 | Base to Base |
| 53.0591 | 1.83E+07 | 1.2049 | 2.30E+06 | 1.6573 | 0.3478 | Base to Base |
| 53.7262 | 1.26E+08 | 8.2723 | 8.93E+06 | 6.4387 | 0.5619 | Base to Base |
| 55.4126 | 1.19E+08 | 7.8049 | 1.12E+07 | 8.068 | 0.4013 | Base to Base |
| 55.8032 | 3.95E+07 | 2.5926 | 4.92E+06 | 3.5508 | 0.3478 | Base to Base |

TABLE 14
MS Fingerprint of extract AV016BaDi(65)04(100).
Peak List:

| | | | Height | % | Width | |
|---|---|---|---|---|---|---|
| Time (min) | Area (counts) | % Area | (cps) | Height | (min) | Baseline Type |
| 1.5858 | 4.95E+08 | 33.2934 | 5.09E+07 | 33.5845 | 0.3211 | Valley |
| 1.6837 | 4.68E+08 | 31.4658 | 4.70E+07 | 30.9815 | 0.2675 | Valley |
| 2.281 | 2.71E+08 | 18.2632 | 2.23E+07 | 14.6901 | 0.3479 | Valley |
| 2.3736 | 1.40E+08 | 9.4462 | 2.07E+07 | 13.6345 | 0.214 | Valley |
| 42.7019 | 3.33E+06 | 0.2239 | 1.08E+06 | 0.7156 | 0.2141 | Base to Base |
| 51.6555 | 9.78E+06 | 0.6583 | 1.30E+06 | 0.8605 | 0.2408 | Base to Base |
| 54.1648 | 7.99E+07 | 5.3782 | 4.28E+06 | 2.8245 | 0.7224 | Base to Base |
| 55.1059 | 3.72E+06 | 0.2501 | 1.06E+06 | 0.697 | 0.107 | Base to Base |
| 57.6108 | 6.57E+06 | 0.4422 | 1.30E+06 | 0.855 | 0.214 | Base to Base |
| 58.0334 | 6.71E+06 | 0.4517 | 9.03E+05 | 0.5959 | 0.2141 | Base to Base |
| 58.9956 | 1.89E+06 | 0.127 | 8.50E+05 | 0.5609 | 0.0802 | Base to Base |

TABLE 15
MS Fingerprint of extract AV016BaSu(65)09(100).
Peak List

| | | % | Height | % | Width | |
|---|---|---|---|---|---|---|
| Time (min) | Area (counts) | Area | (cps) | Height | (min) | Baseline Type |
| 0.6837 | 2.30E+07 | 1.9654 | 2.66E+06 | 2.8378 | 0.3211 | Base to Base |
| 1.7359 | 3.23E+08 | 27.6122 | 2.10E+07 | 22.4515 | 0.6155 | Base to Base |
| 2.1817 | 6.42E+08 | 54.9509 | 5.24E+07 | 55.9679 | 0.3212 | Base to Base |
| 46.2873 | 2.68E+06 | 0.2294 | 1.06E+06 | 1.1283 | 0.0802 | Base to Base |
| 51.3977 | 1.34E+07 | 1.1443 | 1.86E+06 | 1.987 | 0.2408 | Base to Base |
| 51.7551 | 1.14E+07 | 0.9773 | 2.30E+06 | 2.4614 | 0.1605 | Base to Base |
| 53.5882 | 4.82E+07 | 4.1246 | 4.09E+06 | 4.3665 | 0.4281 | Base to Base |
| 56.8025 | 3.06E+07 | 2.6205 | 2.29E+06 | 2.4491 | 0.2943 | Base to Base |
| 57.7937 | 7.45E+07 | 6.3753 | 5.94E+06 | 6.3504 | 0.4549 | Base to Base |

TABLE 16
MS Fingerprint of extract AV016BaSu(65)01(100).
Peak List:

| | | % | Height | % | Width | |
|---|---|---|---|---|---|---|
| Time (min) | Area (counts) | Area | (cps) | Height | (min) | Baseline Type |
| 0.9306 | 6.79E+06 | 0.9084 | 1.15E+06 | 1.8547 | 0.1873 | Base to Base |
| 1.6126 | 3.36E+07 | 4.493 | 1.23E+06 | 1.9756 | 0.2943 | Base to Base |
| 2.2467 | 5.48E+08 | 73.275 | 2.92E+07 | 46.9161 | 0.5887 | Base to Base |
| 2.6959 | 5.61E+06 | 0.7509 | 2.37E+06 | 3.8115 | 0.0803 | Base to Base |
| 8.7292 | 4.57E+06 | 0.6115 | 7.87E+05 | 1.2645 | 0.1605 | Base to Base |
| 22.1814 | 1.19E+07 | 1.5974 | 9.94E+05 | 1.5977 | 0.3478 | Base to Base |
| 22.538 | 5.22E+06 | 0.6979 | 8.29E+05 | 1.3331 | 0.1873 | Base to Base |
| 24.7315 | 5.89E+06 | 0.7887 | 1.07E+06 | 1.7263 | 0.1605 | Base to Base |
| 26.7098 | 3.29E+06 | 0.4408 | 1.01E+06 | 1.6234 | 0.107 | Base to Base |
| 28.9401 | 1.24E+07 | 1.6572 | 1.40E+06 | 2.2533 | 0.2676 | Base to Base |
| 29.316 | 3.87E+06 | 0.5185 | 1.47E+06 | 2.3673 | 0.0803 | Base to Base |
| 33.8775 | 4.43E+06 | 0.5927 | 1.17E+06 | 1.883 | 0.1605 | Base to Base |
| 35.7436 | 4.34E+06 | 0.5805 | 8.92E+05 | 1.4329 | 0.1873 | Base to Base |
| 37.7239 | 6.26E+06 | 0.8374 | 1.17E+06 | 1.8777 | 0.214 | Base to Base |
| 37.8343 | 1.99E+06 | 0.2659 | 7.72E+05 | 1.2402 | 0.107 | Base to Base |
| 39.1183 | 5.39E+06 | 0.7212 | 9.96E+05 | 1.6006 | 0.1606 | Base to Base |
| 44.5179 | 7.33E+06 | 0.9815 | 9.63E+05 | 1.5473 | 0.2676 | Base to Base |
| 49.8635 | 3.66E+06 | 0.4897 | 8.38E+05 | 1.347 | 0.1338 | Base to Base |
| 51.9325 | 3.25E+06 | 0.4342 | 1.23E+06 | 1.9711 | 0.0803 | Base to Base |
| 52.2278 | 3.39E+06 | 0.4532 | 1.25E+06 | 2.0023 | 0.1071 | Base to Base |
| 52.5806 | 1.33E+07 | 1.7735 | 1.73E+06 | 2.7832 | 0.2676 | Valley |
| 52.735 | 1.12E+07 | 1.5033 | 1.69E+06 | 2.7234 | 0.1873 | Valley |
| 53.8915 | 1.27E+07 | 1.706 | 2.22E+06 | 3.5732 | 0.2141 | Base to Base |
| 54.4726 | 7.86E+06 | 1.0524 | 1.51E+06 | 2.4278 | 0.1338 | Base to Base |
| 57.8304 | 1.22E+07 | 1.6307 | 2.45E+06 | 3.9445 | 0.1873 | Base to Base |
| 59.5907 | 5.79E+06 | 0.7754 | 1.11E+06 | 1.7919 | 0.1338 | Base to Base |
| 59.9224 | 3.46E+06 | 0.463 | 7.03E+05 | 1.1303 | 0.1338 | Base to Base |

TABLE 17
MS Fingerprint of extract AV016BaSu(65)01(100).

| | | % | Height | % | Width | |
|---|---|---|---|---|---|---|
| Time (min) | Area (counts) | Area | (cps) | Height | (min) | Baseline Type |
| 0.9354 | 4.30E+06 | 1.2293 | 1.22E+06 | 2.4333 | 0.214 | Base to Base |
| 2.2806 | 1.29E+08 | 36.7178 | 1.08E+07 | 21.6409 | 0.4549 | Base to Base |
| 2.6726 | 4.11E+07 | 11.7354 | 1.62E+07 | 32.297 | 0.0803 | Base to Base |
| 2.9712 | 3.79E+07 | 10.8273 | 7.74E+06 | 15.4562 | 0.1873 | Base to Base |
| 51.3175 | 1.61E+06 | 0.4586 | 6.88E+05 | 1.3736 | 0.0803 | Base to Base |
| 53.8048 | 2.24E+07 | 6.3996 | 1.62E+06 | 3.2379 | 0.3211 | Base to Base |
| 54.3764 | 3.18E+07 | 9.0759 | 2.29E+06 | 4.5779 | 0.5351 | Base to Base |
| 56.2363 | 1.47E+07 | 4.2035 | 1.55E+06 | 3.1017 | 0.3478 | Base to Base |
| 56.9396 | 2.67E+06 | 0.7637 | 1.65E+06 | 3.2962 | 0.0535 | Base to Base |
| 57.4693 | 1.54E+07 | 4.4064 | 2.12E+06 | 4.2359 | 0.2676 | Valley |
| 57.8919 | 4.50E+07 | 12.8633 | 3.30E+06 | 6.595 | 0.5886 | Valley |
| 58.5708 | 4.62E+06 | 1.3192 | 8.79E+05 | 1.7544 | 0.2141 | Base to Base |

TABLE 18
MS Fingerprint of extract AV016BaSu(65)01(100)ng.

| | | % | Height | % | Width | |
|---|---|---|---|---|---|---|
| Time (min) | Area (counts) | Area | (cps) | Height | (min) | Baseline Type |
| 0.2815 | 2.64E+07 | 2.56 | 1.89E+06 | 2.0165 | 0.4014 | Base to Base |
| 0.6177 | 2.15E+07 | 2.0898 | 4.89E+06 | 5.2322 | 0.214 | Base to Base |
| 1.4595 | 6.17E+07 | 5.9816 | 5.53E+06 | 5.911 | 0.3478 | Valley |
| 1.6305 | 2.63E+07 | 2.5549 | 3.33E+06 | 3.5575 | 0.1873 | Valley |
| 2.2215 | 5.21E+08 | 50.5534 | 3.02E+07 | 32.3009 | 0.5352 | Base to Base |
| 4.4158 | 5.86E+06 | 0.5685 | 1.43E+06 | 1.5317 | 0.1605 | Base to Base |
| 10.5664 | 6.14E+06 | 0.5957 | 7.92E+05 | 0.8469 | 0.2408 | Base to Base |
| 23.1719 | 7.20E+06 | 0.6985 | 1.10E+06 | 1.1733 | 0.2141 | Base to Base |
| 26.6471 | 2.14E+06 | 0.2071 | 1.07E+06 | 1.1409 | 0.0803 | Base to Base |
| 28.4723 | 8.22E+06 | 0.7973 | 9.73E+05 | 1.04 | 0.2676 | Base to Base |
| 29.1292 | 3.99E+06 | 0.387 | 7.08E+05 | 0.7574 | 0.1873 | Base to Base |
| 31.5736 | 8.16E+06 | 0.7917 | 8.24E+05 | 0.8813 | 0.2408 | Base to Base |
| 31.6945 | 2.56E+06 | 0.2481 | 7.61E+05 | 0.8141 | 0.107 | Base to Base |
| 33.9826 | 1.00E+07 | 0.9715 | 1.28E+06 | 1.3722 | 0.2943 | Base to Base |
| 36.5048 | 4.65E+06 | 0.4512 | 8.70E+05 | 0.93 | 0.1338 | Base to Base |
| 37.6212 | 3.23E+06 | 0.3131 | 1.19E+06 | 1.2726 | 0.107 | Base to Base |
| 39.8509 | 3.18E+06 | 0.3083 | 7.69E+05 | 0.822 | 0.1338 | Base to Base |
| 42.0646 | 4.16E+06 | 0.4038 | 9.70E+05 | 1.0375 | 0.1605 | Base to Base |
| 42.4115 | 3.05E+06 | 0.2954 | 6.68E+05 | 0.7142 | 0.1338 | Base to Base |
| 42.5886 | 5.36E+06 | 0.5203 | 1.14E+06 | 1.2137 | 0.1338 | Base to Base |
| 43.7214 | 1.66E+06 | 0.1606 | 8.64E+05 | 0.9238 | 0.0803 | Base to Base |
| 49.6266 | 3.44E+06 | 0.3332 | 6.23E+05 | 0.6663 | 0.1605 | Base to Base |
| 49.8477 | 1.80E+06 | 0.175 | 9.30E+05 | 0.9949 | 0.0803 | Base to Base |
| 50.2725 | 2.67E+06 | 0.2585 | 1.22E+06 | 1.3044 | 0.0803 | Base to Base |
| 50.4625 | 2.57E+06 | 0.2489 | 1.25E+06 | 1.3321 | 0.0803 | Base to Base |
| 51.6971 | 9.21E+06 | 0.8936 | 1.33E+06 | 1.4175 | 0.3211 | Base to Base |
| 52.2644 | 3.77E+06 | 0.3661 | 1.08E+06 | 1.1526 | 0.1338 | Base to Base |
| 52.6743 | 1.05E+07 | 1.0181 | 1.27E+06 | 1.3617 | 0.3478 | Base to Base |
| 53.3229 | 1.80E+06 | 0.175 | 7.11E+05 | 0.7607 | 0.0803 | Base to Base |
| 54.0466 | 5.16E+07 | 5.0087 | 5.02E+06 | 5.3685 | 0.4281 | Base to Base |
| 54.5611 | 7.42E+06 | 0.7194 | 1.35E+06 | 1.439 | 0.1873 | Base to Base |
| 55.786 | 3.29E+06 | 0.3187 | 1.22E+06 | 1.304 | 0.107 | Base to Base |
| 56.3205 | 9.29E+06 | 0.9008 | 1.81E+06 | 1.9397 | 0.1605 | Base to Base |
| 56.982 | 1.01E+07 | 0.9748 | 1.91E+06 | 2.0474 | 0.1873 | Base to Base |
| 57.5849 | 6.16E+07 | 5.9742 | 5.18E+06 | 5.5408 | 0.4549 | Valley |
| 57.9722 | 1.07E+08 | 10.4 | 6.43E+06 | 6.8743 | 0.6421 | Valley |
| 59.2956 | 8.01E+06 | 0.7772 | 9.41E+05 | 1.0067 | 0.2676 | Base to Base |

TABLE 19
MS Fingerprint of extract AV016BaSu(65)01(100)g.

| | | % | Height | % | Width | |
|---|---|---|---|---|---|---|
| Time (min) | Area (counts) | Area | (cps) | Height | (min) | Baseline Type |
| 0.2462 | 4.54E+06 | 0.4516 | 1.28E+06 | 1.5367 | 0.1605 | Base to Base |
| 0.7783 | 2.91E+07 | 2.896 | 2.30E+06 | 2.7508 | 0.4281 | Base to Base |
| 1.4229 | 1.44E+08 | 14.3325 | 1.54E+07 | 18.4396 | 0.2676 | Valley |
| 1.6808 | 2.48E+08 | 24.6093 | 1.67E+07 | 20.0141 | 0.4281 | Valley |
| 2.2103 | 5.05E+08 | 50.1865 | 3.12E+07 | 37.4091 | 0.5084 | Base to Base |
| 2.6741 | 2.06E+07 | 2.0484 | 6.02E+06 | 7.2093 | 0.1338 | Base to Base |
| 3.5511 | 4.01E+06 | 0.3982 | 1.49E+06 | 1.7817 | 0.107 | Base to Base |
| 4.8158 | 1.41E+07 | 1.4004 | 2.22E+06 | 2.6599 | 0.2676 | Base to Base |
| 27.5026 | 4.41E+06 | 0.4385 | 1.10E+06 | 1.3192 | 0.2141 | Base to Base |
| 53.9115 | 1.52E+07 | 1.5124 | 1.73E+06 | 2.0716 | 0.3479 | Base to Base |
| 55.1432 | 1.04E+07 | 1.0317 | 1.45E+06 | 1.735 | 0.3478 | Base to Base |
| 56.3186 | 2.19E+06 | 0.2172 | 8.58E+05 | 1.0289 | 0.0803 | Base to Base |
| 57.4751 | 1.54E+06 | 0.153 | 7.41E+05 | 0.8876 | 0.0803 | Base to Base |
| 57.9589 | 3.26E+06 | 0.3241 | 9.65E+05 | 1.1566 | 0.107 | Base to Base |

TABLE 20
MS Fingerprint of extract AV016BaSu(65)04(100).

| | | | Height | % | Width | |
|---|---|---|---|---|---|---|
| Time (min) | Area (counts) | % Area | (cps) | Height | (min) | Baseline Type |
| 1.3402 | 1.14E+08 | 17.1348 | 1.35E+07 | 13.7133 | 0.2676 | Valley |
| 1.6073 | 1.74E+07 | 2.6038 | 1.93E+06 | 1.9612 | 0.1606 | Valley |
| 2.2967 | 1.87E+08 | 28.0404 | 1.01E+07 | 10.3069 | 0.4817 | Base to Base |
| 4.0358 | 4.53E+06 | 0.6786 | 1.01E+06 | 1.0332 | 0.1338 | Valley |
| 4.1691 | 3.10E+06 | 0.4647 | 1.31E+06 | 1.3305 | 0.0803 | Valley |
| 4.385 | 5.02E+06 | 0.7515 | 1.42E+06 | 1.4467 | 0.1338 | Base to Base |
| 4.9989 | 5.94E+06 | 0.8892 | 1.16E+06 | 1.1771 | 0.1605 | Base to Base |
| 5.3019 | 3.46E+06 | 0.5183 | 1.06E+06 | 1.0776 | 0.1338 | Base to Base |
| 5.9113 | 2.78E+06 | 0.4167 | 1.30E+06 | 1.3198 | 0.0803 | Base to Base |
| 6.1778 | 4.49E+06 | 0.6721 | 7.58E+05 | 0.7719 | 0.1873 | Base to Base |
| 6.3704 | 2.71E+06 | 0.4056 | 7.84E+05 | 0.7985 | 0.107 | Base to Base |
| 6.8924 | 6.19E+06 | 0.9275 | 7.75E+05 | 0.7891 | 0.214 | Base to Base |
| 7.2702 | 3.77E+06 | 0.5654 | 1.17E+06 | 1.1885 | 0.107 | Base to Base |
| 7.4357 | 2.04E+06 | 0.3051 | 9.28E+05 | 0.945 | 0.0803 | Base to Base |
| 7.9721 | 4.93E+06 | 0.7391 | 9.22E+05 | 0.939 | 0.1873 | Base to Base |
| 8.8605 | 6.09E+06 | 0.9128 | 7.53E+05 | 0.7667 | 0.2141 | Base to Base |
| 9.4252 | 2.87E+06 | 0.4292 | 7.85E+05 | 0.7996 | 0.1071 | Base to Base |
| 9.789 | 4.10E+06 | 0.6138 | 6.12E+05 | 0.6234 | 0.2676 | Base to Base |
| 10.7492 | 1.94E+06 | 0.2902 | 7.58E+05 | 0.7718 | 0.0803 | Base to Base |
| 13.0858 | 2.60E+06 | 0.3897 | 6.98E+05 | 0.7105 | 0.1094 | Valley |
| 13.1888 | 3.36E+06 | 0.5029 | 7.11E+05 | 0.7238 | 0.1582 | Valley |
| 15.4704 | 4.27E+06 | 0.64 | 1.29E+06 | 1.3121 | 0.1338 | Base to Base |
| 16.5549 | 2.37E+06 | 0.3545 | 8.66E+05 | 0.8822 | 0.0802 | Base to Base |
| 17.1515 | 4.19E+06 | 0.6275 | 1.24E+06 | 1.2608 | 0.107 | Valley |
| 17.2577 | 5.17E+06 | 0.7752 | 9.38E+05 | 0.9557 | 0.1606 | Valley |
| 17.4256 | 5.27E+06 | 0.7899 | 8.05E+05 | 0.8196 | 0.1605 | Base to Base |
| 17.7689 | 5.45E+06 | 0.816 | 1.09E+06 | 1.1149 | 0.1338 | Base to Base |
| 19.3648 | 2.95E+06 | 0.4416 | 8.37E+05 | 0.8528 | 0.1606 | Base to Base |
| 19.5311 | 1.19E+06 | 0.1786 | 5.57E+05 | 0.5667 | 0.0803 | Base to Base |
| 20.302 | 5.74E+06 | 0.8605 | 9.81E+05 | 0.9987 | 0.1605 | Base to Base |
| 23.1422 | 4.84E+06 | 0.7254 | 8.61E+05 | 0.8767 | 0.1605 | Base to Base |
| 23.4055 | 3.51E+06 | 0.5262 | 7.76E+05 | 0.7901 | 0.1345 | Base to Base |
| 25.5012 | 2.20E+06 | 0.3299 | 1.04E+06 | 1.0621 | 0.0803 | Base to Base |
| 25.9266 | 2.59E+06 | 0.3882 | 9.14E+05 | 0.931 | 0.107 | Base to Base |
| 26.7339 | 3.31E+06 | 0.496 | 1.09E+06 | 1.1142 | 0.107 | Base to Base |
| 27.8535 | 3.91E+06 | 0.5853 | 1.02E+06 | 1.0382 | 0.1873 | Base to Base |
| 28.7851 | 2.65E+06 | 0.3968 | 9.95E+05 | 1.0129 | 0.0803 | Valley |
| 28.9276 | 5.31E+06 | 0.7949 | 8.69E+05 | 0.8849 | 0.1873 | Valley |
| 29.0503 | 4.46E+06 | 0.6684 | 1.13E+06 | 1.1496 | 0.1605 | Valley |
| 29.6159 | 2.76E+06 | 0.413 | 1.10E+06 | 1.1195 | 0.107 | Base to Base |
| 31.7858 | 2.66E+06 | 0.3987 | 7.40E+05 | 0.7538 | 0.1338 | Valley |
| 31.9243 | 2.84E+06 | 0.4248 | 5.33E+05 | 0.543 | 0.1605 | Valley |
| 32.5063 | 3.51E+06 | 0.5259 | 1.20E+06 | 1.2204 | 0.107 | Base to Base |
| 33.128 | 3.73E+06 | 0.558 | 7.30E+05 | 0.7436 | 0.2141 | Base to Base |
| 33.3651 | 1.37E+06 | 0.2059 | 5.85E+05 | 0.596 | 0.0803 | Base to Base |
| 34.7807 | 3.81E+06 | 0.5705 | 9.24E+05 | 0.9412 | 0.1338 | Base to Base |
| 35.2633 | 2.05E+06 | 0.3075 | 7.05E+05 | 0.7181 | 0.107 | Base to Base |
| 36.9489 | 5.65E+06 | 0.8458 | 9.78E+05 | 0.9955 | 0.1605 | Base to Base |
| 38.1394 | 2.87E+06 | 0.4303 | 8.43E+05 | 0.8586 | 0.107 | Base to Base |
| 38.6896 | 6.18E+06 | 0.9252 | 1.03E+06 | 1.0486 | 0.2408 | Base to Base |
| 40.5898 | 2.15E+06 | 0.3224 | 5.81E+05 | 0.5917 | 0.1338 | Base to Base |
| 41.5733 | 7.00E+06 | 1.0491 | 9.43E+05 | 0.96 | 0.2408 | Base to Base |
| 42.0674 | 3.46E+06 | 0.5184 | 8.98E+05 | 0.9143 | 0.1338 | Base to Base |
| 42.2718 | 8.14E+06 | 1.2191 | 1.05E+06 | 1.0705 | 0.2676 | Base to Base |
| 42.4327 | 2.55E+06 | 0.3818 | 6.87E+05 | 0.6998 | 0.107 | Base to Base |
| 44.1144 | 7.48E+06 | 1.1211 | 1.08E+06 | 1.1033 | 0.2943 | Base to Base |
| 46.1964 | 6.08E+06 | 0.9103 | 8.94E+05 | 0.9104 | 0.1873 | Base to Base |
| 46.8725 | 3.21E+06 | 0.4811 | 7.64E+05 | 0.7785 | 0.1338 | Base to Base |
| 47.3852 | 1.47E+06 | 0.2207 | 7.06E+05 | 0.7194 | 0.0803 | Base to Base |
| 50.6192 | 1.54E+06 | 0.2307 | 6.74E+05 | 0.6865 | 0.0803 | Base to Base |
| 50.8303 | 3.42E+06 | 0.5122 | 1.03E+06 | 1.0527 | 0.107 | Base to Base |
| 51.2141 | 6.26E+06 | 0.9376 | 1.25E+06 | 1.2757 | 0.1873 | Base to Base |
| 51.531 | 8.74E+06 | 1.3088 | 1.14E+06 | 1.1594 | 0.2408 | Valley |
| 51.6971 | 7.62E+06 | 1.1419 | 1.49E+06 | 1.5193 | 0.1606 | Valley |
| 51.9366 | 3.58E+06 | 0.5358 | 1.13E+06 | 1.146 | 0.1338 | Base to Base |
| 52.5223 | 4.27E+06 | 0.6399 | 1.01E+06 | 1.0277 | 0.1338 | Base to Base |
| 53.2728 | 4.39E+06 | 0.6573 | 7.78E+05 | 0.7923 | 0.1606 | Base to Base |
| 53.8949 | 1.32E+07 | 1.9795 | 1.63E+06 | 1.6568 | 0.3211 | Base to Base |
| 54.4009 | 4.66E+06 | 0.6975 | 1.34E+06 | 1.3633 | 0.107 | Valley |
| 54.5073 | 1.82E+07 | 2.7254 | 1.63E+06 | 1.6624 | 0.2676 | Valley |
| 55.9786 | 4.07E+06 | 0.6101 | 9.34E+05 | 0.9515 | 0.1338 | Base to Base |
| 56.3475 | 1.33E+07 | 1.9928 | 1.13E+06 | 1.1551 | 0.3211 | Valley |
| 56.5847 | 4.61E+06 | 0.6907 | 8.48E+05 | 0.864 | 0.1605 | Valley |
| 56.8275 | 9.26E+06 | 1.3875 | 1.22E+06 | 1.243 | 0.1873 | Valley |
| 57.0145 | 4.88E+06 | 0.7309 | 9.98E+05 | 1.016 | 0.1606 | Valley |
| 57.1188 | 2.45E+06 | 0.3669 | 7.75E+05 | 0.7891 | 0.107 | Base to Base |
| 57.7155 | 6.35E+06 | 0.952 | 1.11E+06 | 1.1327 | 0.1606 | Valley |
| 57.8152 | 9.52E+06 | 1.4268 | 1.38E+06 | 1.4031 | 0.2141 | Valley |

TABLE 21
MS Fingerprint of extract AV016BaSu(65)06(100).

| | | % | Height | % | Width | |
|---|---|---|---|---|---|---|
| Time (min) | Area (counts) | Area | (cps) | Height | (min) | Baseline Type |
| 1.3425 | 1.34E+08 | 15.6624 | 2.35E+07 | 34.2569 | 0.2141 | Base to Base |
| 2.3068 | 6.22E+08 | 72.7416 | 3.24E+07 | 47.2835 | 0.5886 | Base to Base |
| 4.4357 | 5.31E+06 | 0.6201 | 1.63E+06 | 2.3849 | 0.1605 | Base to Base |
| 5.2161 | 2.96E+06 | 0.3463 | 1.29E+06 | 1.8752 | 0.107 | Base to Base |
| 5.6491 | 9.55E+06 | 1.1164 | 1.58E+06 | 2.3015 | 0.1873 | Base to Base |
| 6.6417 | 9.07E+06 | 1.0594 | 2.04E+06 | 2.9753 | 0.1605 | Base to Base |
| 17.6827 | 1.27E+07 | 1.4888 | 1.34E+06 | 1.954 | 0.4281 | Base to Base |
| 54.1984 | 5.00E+07 | 5.8447 | 3.32E+06 | 4.8386 | 0.4548 | Base to Base |
| 57.3586 | 9.59E+06 | 1.1203 | 1.46E+06 | 2.1301 | 0.1873 | Base to Base |

TABLE 22
MS Fingerprint of extract AV016BaSu(105)08(100).

| | | % | Height | % | Width | |
|---|---|---|---|---|---|---|
| Time (min) | Area (counts) | Area | (cps) | Height | (min) | Baseline Type |
| 2.0569 | 2.14E+08 | 27.1727 | 2.15E+07 | 36.3993 | 0.2677 | Base to Base |
| 3.0165 | 5.13E+08 | 65.1587 | 2.56E+07 | 43.3466 | 0.7224 | Base to Base |
| 4.4937 | 1.06E+07 | 1.3463 | 2.07E+06 | 3.4978 | 0.1873 | Base to Base |
| 4.8116 | 9.88E+06 | 1.255 | 1.36E+06 | 2.304 | 0.2141 | Base to Base |
| 7.1128 | 9.55E+06 | 1.2133 | 1.79E+06 | 3.0334 | 0.1606 | Base to Base |
| 20.2582 | 6.97E+06 | 0.8856 | 1.05E+06 | 1.7785 | 0.1873 | Base to Base |
| 21.0285 | 2.37E+06 | 0.3008 | 8.07E+05 | 1.3647 | 0.107 | Base to Base |
| 21.9949 | 5.91E+06 | 0.751 | 1.41E+06 | 2.3921 | 0.1605 | Base to Base |
| 26.1397 | 4.67E+06 | 0.5933 | 1.60E+06 | 2.7139 | 0.1338 | Base to Base |
| 28.479 | 1.04E+07 | 1.3233 | 1.87E+06 | 3.1697 | 0.1873 | Base to Base |

TABLE 23
MS Fingerprint of extract AV016FrDi(65)04(100).

| | | % | Height | % | Width | |
|---|---|---|---|---|---|---|
| Time (min) | Area (counts) | Area | (cps) | Height | (min) | Baseline Type |
| 1.6028 | 1.52E+09 | 29.4877 | 6.12E+07 | 29.3519 | 0.6423 | Valley |
| 2.2961 | 3.50E+09 | 68.0186 | 1.30E+08 | 62.3426 | 0.99 | Valley |
| 4.0708 | 3.33E+06 | 0.0647 | 1.35E+06 | 0.6474 | 0.107 | Base to Base |
| 4.8702 | 1.71E+07 | 0.3327 | 2.48E+06 | 1.1882 | 0.2675 | Base to Base |
| 5.4024 | 9.12E+06 | 0.1773 | 2.48E+06 | 1.1914 | 0.1338 | Base to Base |
| 6.2587 | 4.84E+06 | 0.0941 | 1.53E+06 | 0.7348 | 0.107 | Base to Base |
| 47.0653 | 5.85E+07 | 1.1379 | 2.31E+06 | 1.1097 | 0.6689 | Base to Base |
| 48.8324 | 4.52E+06 | 0.0879 | 1.54E+06 | 0.7369 | 0.1338 | Base to Base |
| 49.5832 | 5.13E+06 | 0.0998 | 1.18E+06 | 0.5668 | 0.1338 | Base to Base |
| 51.2074 | 3.34E+06 | 0.065 | 1.38E+06 | 0.6601 | 0.0802 | Base to Base |
| 51.9303 | 6.03E+06 | 0.1171 | 1.52E+06 | 0.7281 | 0.1338 | Base to Base |
| 57.0683 | 1.63E+07 | 0.3173 | 1.55E+06 | 0.7421 | 0.3211 | Base to Base |

TABLE 24
MS Fingerprint of extract AV016FrSu(105)08(100).
Peak List:

| | | % | Height | % | Width | |
|---|---|---|---|---|---|---|
| Time (min) | Area (counts) | Area | (cps) | Height | (min) | Baseline Type |
| 1.9597 | 2.64E+08 | 29.489 | 2.96E+07 | 45.0514 | 0.2677 | Base to Base |
| 3.1068 | 6.21E+08 | 69.4326 | 3.28E+07 | 49.9315 | 0.6689 | Base to Base |
| 5.6942 | 4.34E+06 | 0.485 | 1.88E+06 | 2.8549 | 0.0802 | Base to Base |
| 22.1023 | 5.31E+06 | 0.5934 | 1.42E+06 | 2.1622 | 0.1605 | Base to Base |

TABLE 25
IC₅₀ values of antioxidation potential of T. arjuna extracts from different plant parts.

| | | Extraction | IC₅₀ |
|---|---|---|---|
| Plant Part | Extract-ID | Description | (μg/ml) |
| 1. Bark | AV016BaDi(65)04(100) | Direct 100% ethanol | 26 |
| 2. Bark | AV016BaDi(28)04(20) | Direct 20% ethanol | 24 |
| 3. Bark | AV016BaSu(65)01(100)g | Successive 100% acetone | 26 |
| 4. Bark | AV016BaSu(65)01(100)ng | Successive 100% acetone | 46 |
| 5. Bark | AV016BaSu(65)01(100) | Successive 100% acetone | 24 |
| 6. Bark | AV016BaSu(65)04(100) | Successive 100% ethanol | 37 |
| 7. Bark | AV016BaSu(65)06(100) | Successive 100% methanol | 34 |
| 8. Bark | AV016BaSu(105)08(100) | Successive 100% water | 46 |
| 9. Bark | AV016BaSu(65)09(100) | Successive 100% ethyl acetate | 53 |
| 10. Fruit | AV016FrDi(65)04(100) | Direct 100% ethanol | 34 |
| 11. Fruit | AV016FrDi(105)08(100) | Successive 100% water | 39 |
| 12. Ascorbic acid | | | 26 |
| (positive control) | | | |

TABLE 26
Anti-microbial activity of Terminalia arjuna bark successive extracts:

| | | Extracts | | | | | | | |
|---|---|---|---|---|---|---|---|---|---|
| | | AV016BaSu(65)09 | | AV016BaSu(65)01 | | AV016BaSu(65)04 | | AV016BaSu(65)07 | |
| Sr. | | (100) | | (100) | | (100) | | (100) | |
| No. | Organism | 1 mg/ml | 5 mg/ml | 1 mg/ml | 5 mg/ml | 1 mg/ml | 5 mg/ml | 1 mg/ml | 5 mg/ml |
| | Gram Negative | | | | | | | | |
| 1. | E. coli | +++ | +++ | +++ | +++ | +++ | +++ | +++ | +++ |
| 2. | P. aeruginosa | +++ | +++ | +++ | +++ | +++ | +++ | +++ | +++ |
| 3. | K. pneumoniea | +++ | − | +++ | ++ | +++ | +++ | +++ | +++ |
| 4. | B. bronchiseptica | − | − | + | − | +++ | − | +++ | − |
| | Gram Positive | | | | | | | | |
| 5. | S. aureus | − | − | − | − | − | − | − | − |
| 6. | S. fecalis | − | − | − | − | +++ | − | +++ | − |
| 7. | M. luteus | +++ | − | +++ | − | +++ | − | +++ | − |
| 8. | B. subtilis | +++ | − | +++ | +++ | +++ | +++ | +++ | +++ |
| 9. | B. cereus | +++ | − | +++ | +++ | +++ | +++ | +++ | +++ |
| 10. | B. pumilus | +++ | − | +++ | +++ | +++ | +++ | +++ | +++ |
| 11. | S. epidermidis | +++ | − | +++ | +++ | +++ | +++ | +++ | +++ |

| | | Extracts | | | | |
|---|---|---|---|---|---|---|
| | | AV016BaSu(65)08 | | Control | | |
| Sr. | | (100) | | | LB + DMSO | LB + Ciprofloxacin |
| No. | Organism | 1 mg/ml | 5 mg/ml | LB | (5%) | (2 μg/ml) |
| | Gram Negative | | | | | |
| 1. | E. coli | +++ | +++ | +++ | +++ | − |
| 2. | P. aeruginosa | +++ | +++ | +++ | +++ | − |
| 3. | K. pneumoniea | +++ | +++ | +++ | +++ | − |
| 4. | B. bronchiseptica | +++ | − | +++ | +++ | − |
| | Gram Positive | | | | | |
| 5. | S. aureus | − | − | +++ | +++ | − |
| 6. | S. fecalis | +++ | − | +++ | +++ | − |
| 7. | M. luteus | +++ | − | +++ | +++ | − |
| 8. | B. subtilis | +++ | +++ | +++ | +++ | − |
| 9. | B. cereus | +++ | +++ | +++ | +++ | − |
| 10. | B. pumilus | +++ | +++ | +++ | +++ | − |
| 11. | S. epidermidis | +++ | +++ | +++ | +++ | − |

+++; abundant growth,
++; growth;
+ less growth;
−, no growth

TABLE 27
Anti-bacterial activity of Terminalia arjuna fruit extracts:

| | | Extracts | | | | | | |
|---|---|---|---|---|---|---|---|---|
| | | AV016FrDi(65)04 | | AV016FrSu(65)08 | | Control | | |
| Sr. | | (100) | | (105) | | | LB + DMSO | LB + Ciprofloxacin |
| No. | Organism | 1 mg/ml | 5 mg/ml | 1 mg/ml | 5 mg/ml | LB | (5%) | (2 μg/ml) |
| | Gram Negative | | | | | | | |
| 1. | E. coli | +++ | ++ | +++ | +++ | +++ | +++ | − |
| 2. | P. aeruginosa | +++ | ++ | +++ | +++ | +++ | +++ | − |
| 3. | K. pneumoniea | +++ | + | +++ | +++ | +++ | +++ | − |
| 4. | B. bronchiseptica | − | − | +++ | − | +++ | +++ | − |
| | Gram Positive | | | | | | | |
| 5. | S. aureus | +++ | ++ | +++ | +++ | +++ | +++ | − |
| 6. | S. fecalis | +++ | ++ | +++ | +++ | +++ | +++ | − |
| 7. | M. luteus | +++ | ++ | +++ | +++ | +++ | +++ | − |
| 8. | B. cereus | +++ | − | +++ | +++ | +++ | +++ | − |
| 9. | B. pumilus | +++ | − | +++ | +++ | +++ | +++ | − |
| 10. | S. epidermidis | +++ | − | +++ | +++ | +++ | +++ | − |

+++; abundant growth,
++; growth;
+ less growth;
−, no growth

1. A method for treating a disease selected from the group comprising cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract in a mammal, which comprises administering to the said mammal an effective non-toxic amount of at least an extract from Terminalia arjuna selected from those as defined in Tables 1-24.

2. A method for treating infectious diseases in a mammal, which comprises administering to the said mammal an effective non-toxic amount of at least an extract from Terminalia arjuna selected from those as defined in Tables 1-24.

3. A method according to claim 1 wherein the disease is selected from the group comprising cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract and the extract is selected from the group consisting of AV016BaDi(65)04(1100), AV016BaDi(28)04(20), AV016BaSu(65)09(100), AV016BaSu(65)01 (100), AV016BaSu(65)01 (100)g, AV016BaSu(65)01 (100)ng, AV016BaSu(65)04(1100), AV016BaSu(65)06(1100), AV016BaSu(105)08(100), AV016FrDi(65)04(100) and AV016FrSu(105)08(100), or a combination of two or more thereof.

4. A method according to claim 2 wherein the disease is any infectious disease and the extract is selected from the group consisting of AV016BaSu(65)09(100), AV016BaSu(65)01 (100), AV016BaSu(65)04(1100), AV016BaSu(65)06(1100), AV016BaSu(105)08(100), AV016FrDi(65)04(100) and AV016FrSu(105)08(100), or a combination of two or more thereof.

5. A method according claim 1 wherein the said treatment is a prophylactic treatment.

6. A pharmaceutical formulation for use in the treatment of a disease selected from the group consisting of cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract, comprising at least one extract isolated from Terminalia arjuna, and selected from those listed in Tables 1-24 in admixture with a pharmaceutically acceptable carrier.

7. A pharmaceutical formulation for use in the treatment of any infectious disease, comprising at least one extract isolated from Terminalia arjuna, and selected from those listed in Tables 1-24 in admixture with a pharmaceutically acceptable carrier.

8. A formulation according to claim 6 for use in the treatment of a disease selected from the group consisting of cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract, comprising at least one extract selected from the group consisting of AV016BaDi(65)04(100), AV016BaDi(28)04(20), AV016BaSu(65)09(1100), AV016BaSu(65)01 (100), AV016BaSu(65)01 (100)g, AV016BaSu(65)01(100)ng, AV016BaSu(65)04(1100), AV016BaSu(65)06(1100), AV016BaSu(105)08(100), AV016FrDi(65)04(100) and AV016FrSu(105)08(100), or a combination of two or more thereof.

9. A formulation according to claim 7 for use in the treatment of any infectious disease, comprising at least one extract selected from the group consisting of, AV016BaSu(65)09(1100), AV016BaSu(65)01(100), AV016BaSu(65)04(100), AV016BaSu(65)06(1100), AV016BaSu(1105)08(100), AV016FrDi(65)04(100) and AV016FrSu(105)08(100), or a combination of two or more thereof.

10. A formulation according to claim 6 for prophylactic use.

11. A method for the preparation of a pharmaceutical formulation comprising bringing into association at least an extract of the invention, and a pharmaceutically acceptable carrier therefore.

12. An extract from Terminalia arjuna selected from the group consisting of the extracts having the HPLC and/or MS characteristics shown in Tables 1-24.

13. A comestible comprising at least an extract from Terminalia arjuna selected from the group consisting of the extracts having the HPLC and/or MS characteristics shown in Tables 1-24.

14. A comestible according to claim 13 comprising at least an extract for use in the prophylaxis of a disease selected from the group comprising cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract.

15. A comestible according to claim 13 comprising at least an extract for use in the prophylaxis of any infectious disease.

16. Use of an extract selected from the group consisting of the extracts having the HPLC and/or MS characteristics shown in Tables 1-24 for the preparation of a medicament for the treatment of disease selected from the group consisting of cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract.

17. Use according to claim 16 of an extract selected from the group consisting of AV016BaDi(65)04(100), AV016BaDi(28)04(20), AV016BaSu(65)09(1100), AV016BaSu(65)01 (100), AV016BaSu(65)01 (100)g, AV016BaSu(65)01 (100)ng, AV016BaSu(65)04(1100), AV016BaSu(65)06(1100), AV016BaSu(105)08(100), AV016FrDi(65)04(100) and AV016FrSu(105)08(100), for the preparation of a medicament for the treatment or prophylaxis of disease selected from the group consisting of cardiovascular disease, diabetes, degenerative neurological diseases, cancer, age related diseases like amyloidosis, acute pancreatitis, arthritis, atherosclerosis, cancer, heart disease, inflammatory bowel disease, myocardial infarction, senile dementia, retinal degeneration and senile cataract.

18. Use of an extract selected from the group consisting of the extracts having the HPLC and/or MS characteristics shown in Tables 1-24 for the preparation of a medicament for the treatment of any infectious disease.

19. Use according to claim 18 of an extract selected from the group consisting of AV016BaSu(65)09(1100), AV016BaSu(65)01 (100), AV016BaSu(65)04(1100), AV016BaSu(65)06(100), AV016BaSu(105)08(100), AV016FrDi(65)04(100) and AV016FrSu(105)08(100), for the preparation of a medicament for the treatment or prophylaxis of any infectious disease.

20. A method according claim 2 wherein the said treatment is a prophylactic treatment.

21. A formulation according to claim 7 for prophylactic use.
